(19) United States
(12) Patent Application Publication
(10) Pub. No.: US 2011/0014216 A1
(43) Pub. Date: Jan. 20, 2011

(54) Drug Transfer Based on Coenzyme A and Acyl Carrier Protein

(75) Inventors: Andreas Brecht, Muttenz (CH); Florent Beaufils, Rosenau (FR)

Correspondence Address: HARRIET M. STRIMPEL, D. Phil., New England Biolabs, Inc., 240 COUNTY ROAD, IPSWICH, MA, 01938-2723, US

(73) Assignee: COVALYS BIOSCIENCES AG, Witterswil (CH)

(21) Appl. No.: 12/681,450

(22) PCT Filed: Oct. 2, 2008
(86) PCT No.: PCT/EP08/63207

§ 371 Date: Oct. 5, 2010

(30) Foreign Application Priority Data
Oct. 3, 2007 (EP) 07117802.4

(51) Int. Cl. A61K 39/44; C07K 19/00; C07H 19/207; C07H 21/02; C07K 17/10; A61P 35/00

(52) U.S. Cl. 424/178.1; 530/391.3; 530/391.7; 536/26.23; 536/24.5; 530/408

(57) ABSTRACT

The invention relates to coenzyme A (CoA) type compounds carrying one or more drug entities and optionally a label detectable by a fluorescence detector, magnetic resonance imaging (MRI), positron emission tomography (PET) or scintigraphy, and/or a functional group which can be transformed into a drug or a detectable label. The invention further relates to a molecular shuttle which is a fusion protein comprising a proteinaceous binding entity directed to a target and an acyl carrier protein (ACP) or a fragment thereof, and carrying one or more drug entities. The proteinaceous binding entity is designed to bind to a target structure in vitro or in vivo, for example a cellular receptor.

### FIELD OF THE INVENTION

The present invention relates to compounds based on Coenzyme A suitable for transferring a drug or a drug together with a detectable label first to fusion proteins comprising Acyl Carrier Protein and a proteinaceous binder directed to a target, and then to the target, and corresponding methods.

### BACKGROUND OF THE INVENTION

There is a constant need for improved techniques to direct a drug to the desired site of action. Desirable features of molecular shuttles, i.e. compounds for directing drugs to the desired site of action, are low immunogenicity, high target specificity and high avidity. Standard molecular shuttles for such applications are antibodies, in particular humanized antibodies carrying the corresponding drug.

In International Patent Application WO2004/104588 it was demonstrated that a label may be transferred to a protein of interest using the combination of an acyl carrier protein (ACP) or a fragment thereof, a labeled coenzyme A (CoA) type substrate and a holo-acyl carrier protein synthase (ACPS) or a homologue thereof. The ACPS transfers the label from CoA to a fusion protein comprising the protein of interest and an ACP or ACP fragment.

### SUMMARY OF THE INVENTION

The invention relates to coenzyme A (CoA) type compounds carrying one or more drug entities and optionally a label detectable by a fluorescence detector, magnetic resonance imaging (MRI), positron emission tomography (PET) or scintigraphy, and/or a functional group which can be transformed into a drug or a detectable label.

The one or more drugs and the optional detectable label and/or functional group are bound to CoA by a “linker”, which is a linking unit consisting of 1 to 300 carbon atoms, wherein up to a third of the carbon atoms may be replaced by oxygen atoms and/or nitrogen atoms and/or one or more carbon atoms may be replaced by sulfur atoms, may be linear or branched and/or comprise double bonds, triple bonds, carbocycles or heterocycles, and may carry further substituents, in particular an oxo group on a carbon atom adjacent to a nitrogen atom or an oxygen atom.

The invention further relates to a molecular shuttle which is a fusion protein comprising a proteinaceous binding entity directed to a target and an acyl carrier protein (ACP) or a fragment thereof, and carrying one or more drug entities and optionally a label detectable by a fluorescence detector, magnetic resonance imaging (MRI), positron emission tomography (PET) or scintigraphy and/or a functional group which can be transformed into a drug or a detectable label.

The proteinaceous binding entity is designed to bind to a target structure in vitro or in vivo, for example a cellular receptor. A particular binding entity is an antibody or antibody fragment.

Moreover the invention relates to a method of reacting a fusion protein comprising a proteinaceous binding entity directed to a target and an acyl carrier protein (ACP) or a fragment thereof with a coenzyme A (CoA) type compound carrying one or more drug entities and optionally a label detectable by a fluorescence detector, magnetic resonance imaging (MRI), positron emission tomography (PET) or scintigraphy, and a holo-acyl carrier protein synthase (ACPS) or a homologue thereof so that the ACPS transfers the one or more drug entities and the optional detectable label to the fusion protein.

Furthermore the invention relates to pharmaceutical compositions comprising the molecular shuttles as defined hereinbefore, and to a method of treatment comprising administering a molecular shuttle or a pharmaceutical composition comprising a molecular shuttle as defined hereinbefore.

### DETAILED DESCRIPTION OF THE INVENTION

The invention relates to coenzyme A (CoA) type compounds carrying one or more drug entities and optionally a label detectable by a fluorescence detector, magnetic resonance imaging (MRI), positron emission tomography (PET) or scintigraphy, and/or a functional group which can be transformed into a drug or a detectable label.

In particular, the invention relates to compounds of formula

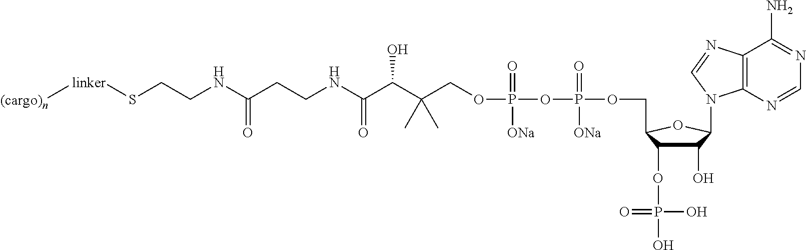

wherein
“cargo” is a drug, whereby several “cargo” entities may be the same or different drug, one or more drugs and a label detectable by a fluorescence detector, magnetic resonance imaging (MRI), positron emission tomography (PET) or scintigraphy, or one or more drugs and a functional group which can be transformed into a drug or a detectable label;
n is 1 or more, for example 1, 2, 3 or 4, in particular 1 or 2; and
“linker” is a linking unit consisting of 1 to 300 carbon atoms, wherein up to a third of the carbon atoms may be replaced by oxygen atoms and/or nitrogen atoms and/or one or more carbon atoms may be replaced by sulfur atoms, may be linear or branched and/or comprise double bonds, triple bonds, carbocycles or heterocycles, and may carry further substituents, in particular an oxo group on a carbon atom adjacent to a nitrogen atom or an oxygen atom.

However, the invention is not restricted to the compounds of the formula shown above, but includes compounds with substitutions of the purine part, modifications of the sugar moiety or of the panthetheinylic acid moiety, for example, a modification wherein the amidoethylthio group connected to the linker is replaced by an amido or ester function.

Drugs considered as cargo are drugs which are more effective when transported to a particular site within the body. Examples are drugs which should interact with a particular cellular receptor or other entity, for example cytotoxic drugs which should be transported to the site of cancer cells. In particular, such drugs considered in the present application are chlorambucil, podophyllotoxin, methotrexate, topotecan hydrochloride, and camptothecin. Modified derivatives of vinca alkaloids (such as vincristine, vinblastine, vinorelbine and vindesine) and taxanes (such as paclitaxel, docetaxel, taxotere) are also considered.

Further examples of drugs are radioactive materials with short half life and limited penetration depth of the radiation emitted upon decay of the radioactive isotope, typically short lived emitters of alpha-radiation, for example derivatives of ^{99m}-Tc, ¹¹¹In, ²¹¹At, and ²¹²Bi from ²¹²Pb (see for example Fritzberg A. R. in Journal of Nuclear Medicine 39:20 N, 1998).

Other examples of drugs considered are oligonucleotides, e.g. DNA or RNA strands with the ability to have a significant effect on the situation of the targeted cell, but also nucleic acid derivatives and analogues, e.g. compounds in which the sugar phosphate backbone is replaced by other units, such as e.g. amino acids (such compounds are denoted PNA and are described in WO 92/20702), more preferably RNAi, and most preferably precursors of siRNA or siRNA itself, preferably with the potential to downregulate a particular protein of interest, or to stop a certain metabolic pathway, e.g. to enhance the effect of a co-administered cytotoxic drug.

Labels detectable by a fluorescence detector, magnetic resonance imaging (MRI), positron emission tomography (PET) or scintigraphy as cargo are, for example, fluorophores, more preferably NIR fluorophores excitable between 650 nm and 950 nm, which are detectable in vitro and in vivo by fluorescence detection systems. Further detectable labels are iron oxide particles and other groups with high contrast for MRI imaging applications. Further detectable labels considered are radiopharmaceutical labels used for imaging, for example on the basis of technetium, Tc-99m.

A functional group which can be transformed into a drug or a detectable label considered as cargo is a protected reactive group, preferably one that can be easily deprotected before use and shows high reactivity towards reaction partners introducing an unstable drug, such as radioactive materials with short half life suitably complexed, or introducing a short-lived detectable radiopharmaceutical label for imaging purposes. Examples of such functional groups are protected amino functions, e.g. trifluoracetamides, easily deprotected and able to react with activated carboxylic acids. Another example is a lipoic acid derivative, which may be easily loaded with ⁷²As³⁺, which reaction is based on the reduction of a disulfide bond in the lipoic acid unit to a dithiol as described in U.S. Pat. No. 5,914,096. Once the dithiol is formed, addition of As³⁺ will result in formation of a covalently bound arsenic through two sulfur-arsenic bonds.

A further functional group which can be transformed into a drug or a detectable label considered as cargo is a group being able to complex radioactive metal isotopes, for example diethylenetriaminepentaacetic acid (DTPA), which is a widely used as organic ligand in magnetic resonance imaging (MRI) and positron emission tomography (PET).

Several “cargo” entities may be the same or different drug, detectable label or functional group. For example one cargo entity may be a cytotoxic drug and another cargo entity a siRNA increasing sensitivity towards the cytotoxic drug, or one cargo may be a drug and another cargo a detectable label, e.g. a fluorescent label.

Preferred cargo entities are drugs and detectable labels as defined hereinbefore.

The linker is preferably a flexible linker connecting the CoA entity to the one or more drugs and the optional detectable label or functional group. Linker units are chosen in the context of the envisioned application, i.e. the transfer of the cargo to an ACP fusion protein. They also increase the solubility of the CoA entity and cargo in the appropriate solvent. The linkers used are chemically stable under the conditions of the actual application. The linkers do not interfere with the reaction of the CoA type compounds with ACP nor with the function of the cargo.

In particular, a linker is a straight or branched chain alkylene group with 1 to 300 carbon atoms, wherein optionally

(a) one or more carbon atoms are replaced by oxygen, in particular wherein every third carbon atom is replaced by oxygen, e.g. a polyethyleneoxy group with 1 to 100 ethyleneoxy units;
(b) one or more carbon atoms are replaced by nitrogen carrying a hydrogen atom or further substituent, representing an amine function, or, in the case that the adjacent carbon atom is substituted by oxo, an amide function —NH—CO—, or, if two adjacent carbon atoms are replaced by nitrogen atoms, a hydrazine function —NH—NH— or a carbonylhydrazine function —NH—NH—CO—;
(c) one or more carbon atoms are replaced by oxygen, and the adjacent carbon atoms are substituted by oxo, representing an ester function —O—CO—;
(d) the bond between two adjacent carbon atoms is a double or a triple bond, representing a function —CH═CH— or —C≡C—, or the bond between a carbon and a nitrogen atom is a double bond representing an imine —C(R)═N— or a hydrazone —C(R)═N—NH—, or the bond between two adjacent nitrogen atoms is a double bond representing a diazo group —N═N—;
(e) one or more carbon atoms are replaced by a phenylene, a saturated or unsaturated cycloalkylene, a saturated or unsaturated bicycloalkylene, a bridging heteroaromatic or a bridging saturated or unsaturated heterocyclyl group;
(f) one or more carbon atoms are replaced by a sulfur atom, representing a thioether or, if two adjacent carbon atoms are replaced by sulfur atoms, a disulfide linkage —S—S—;
or a combination of two or more, especially two or three, alkylene and/or modified alkylene groups as defined under (a) to (f) hereinbefore, optionally containing substituents.

Substituents considered are e.g. lower alkyl, e.g. methyl, hydroxy, lower alkoxy, e.g. methoxy, lower acyloxy, e.g. acetoxy, amino, lower acylamino, e.g. acetylamino or trifluoroacetylamino, halogenyl, e.g. chloro, or oxo.

Further substituents considered are e.g. those obtained when an α-amino acid, in particular a naturally occurring α-amino acid, is incorporated in the linker, wherein carbon atoms are replaced by amide functions —NH—CO— as defined under (b). In such a linker, part of the carbon chain of the alkylene group is replaced by a group —(NH—CHR—CO)ₓ— wherein x is between 1 and 100 and R represents a varying residue of an α-amino acid.

A phenylene group replacing carbon atoms as defined under (e) hereinbefore is e.g. 1,2-, 1,3-, or preferably 1,4-phenylene. A saturated or unsaturated cycloalkylene group replacing carbon atoms as defined under (e) hereinbefore is derived from cycloalkyl with 3 to 7 carbon atoms, preferably from cyclopentyl or cyclohexyl, and is e.g. 1,2- or 1,3-cyclopentylene, 1,2-, 1,3-, or preferably 1,4-cyclohexylene, or also 1,4-cyclohexylene being unsaturated, e.g. in 1- or in 2-position. A saturated or unsaturated bicycloalkylene group replacing carbon atoms as defined under (e) hereinbefore is derived from bicycloalkyl with 7 or 8 carbon atoms, and is e.g. bicyclo[2.2.1]heptylene or bicyclo[2.2.2]octylene, preferably 1,4-bicyclo[2.2.1]heptylene optionally unsaturated in 2-position or doubly unsaturated in 2- and 5-position, and 1,4-bicyclo[2.2.2]octylene optionally unsaturated in 2-position or doubly unsaturated in 2- and 5-position. A bridging heteroaromatic group replacing carbon atoms as defined under (e) hereinbefore is e.g. triazolidene, preferably 1,4-triazolidene, or isoxazolidene, preferably 3,5-isoxazolidene. A bridging saturated or unsaturated heterocyclyl group replacing carbon atoms as defined under (e) hereinbefore is e.g. derived from an unsaturated heterocyclyl group, e.g. 3,5-isoxazolidinene, or a fully saturated heterocyclyl group with 3 to 12 atoms, 1 to 3 of which are heteroatoms selected from nitrogen, oxygen and sulfur, e.g. pyrrolidinediyl, piperidinediyl, tetrahydrofuranediyl, dioxanediyl, morpholinediyl or tetrahydrothiophenediyl, preferably 2,5-dioxopyrrolidine-1,3-diyl(succinimido), 2,5-tetrahydrofuranediyl or 2,5-dioxanediyl. A particular heterocyclyl group considered is a saccharide moiety, e.g. an α- or β-furanosyl or α- or β-pyranosyl moiety, or a succinimido group.

A linker is preferably a straight chain or a doubly or triply branched chain alkylene group with 6 to 25 carbon atoms optionally comprising one or more, for example 1 to 6 amide functions —NH—CO—, or a straight chain or a doubly or triply branched chain polyethylene glycol group with 3 to 100 ethyleneoxy units, optionally comprising one or more, for example 1 to 6 amide functions —NH—CO—, and optionally thioether function and a succinimido group, i.e. a nitrogen containing five-membered heterocycle bound to the alkylene chain through the nitrogen atom and a carbon atom, and further substituted by two oxo groups at the two carbon atoms next to nitrogen. The thioether function is preferably connected to the succinimido group. Further preferred is a straight chain or branched linker comprising one or more polyethylene glycol groups of 3 to 12 ethylene glycol units and alkylene groups wherein carbon atoms are replaced by amide bonds, and further carrying substituted amino and hydroxy functions and/or thioether and succinimido groups. Other preferred branched linkers have dendritic (tree-like) structures wherein amine, carboxamide, ether and/or thioether functions replace carbon atoms of an alkylene group.

A particularly preferred linker is a doubly or triply branched chain alkylene group with 6 to 25 carbon atoms comprising one or more, for example 1 to 6 amide functions —NH—CO— and/or thioether and succinimido groups, or a doubly or triply branched chain polyethylene glycol group with 3 to 36 ethyleneoxy units comprising one or more, for example 1 to 6 amide functions —NH—CO— and/or thioether and succinimido groups.

Other preferred linkers are those comprising a disulfanyl function or a hydrazone function, for example a carbonylhydrazone function.

In particular the linker may contain a structure improving the endosomal release of cargo, taken up by a cell through internalization of the shuttle according to the invention.

Preferred are intracellularly labile linkers, such as linkers comprising a disulfanyl function, a hydrazone or carbonylhydrazone function, carboxylic ester functions (which may be cleaved by intracellular esterases) or synthetic peptide functions (prone to degradation by intracellular peptidases and proteases). Such intracellular cleavage will promote release of the cargo from the endosomes or lysosomes, which is particularly preferred if the cargo is a drug.

Lower alkyl is alkyl with 1 to 7, preferably from 1 to 4 C atoms, and is linear or branched; preferably, lower alkyl is butyl, such as n-butyl, sec-butyl, isobutyl, tert-butyl, propyl, such as n-propyl or isopropyl, ethyl or methyl. Most preferably, lower alkyl is methyl.

In lower alkoxy, the lower alkyl group is as defined hereinbefore. Lower alkoxy denotes preferably n-butoxy, tert-butoxy, iso-propoxy, ethoxy, or methoxy, in particular methoxy.

In lower acyloxy or acylamino, lower acyl has the meaning of formyl or lower alkylcarbonyl wherein lower alkyl is defined as hereinbefore. Lower acyloxy denotes preferably n-butyroxy, n-propionoxy, iso-propionoxy, acetoxy, or formyloxy, in particular acetoxy. Lower acylamino is preferably acetylamino.

Halogen is fluoro, chloro, bromo or iodo, in particular chloro.

The invention further relates to a molecular shuttle which is a fusion protein comprising a proteinaceous binding entity directed to a target and an acyl carrier protein (ACP) or a fragment thereof, and carrying one or more drug entities and optionally a label detectable by a fluorescence detector, magnetic resonance imaging (MRI), positron emission tomography (PET) or scintigraphy and/or a functional group which can be transformed into a drug or a detectable label.

In particular the invention further relates to a molecular shuttle of the formula (cargo)ₙ-linker-(fusion protein) wherein the fusion protein comprises a proteinaceous binding entity directed to a target and an acyl carrier protein (ACP) or a fragment thereof. The molecular shuttle carries one or more drug entities and optionally a label detectable by a fluorescence detector, magnetic resonance imaging (MRI), positron emission tomography (PET) or scintigraphy, and/or a functional group which can be transformed into a drug or a detectable label.

The proteinaceous binding entity is designed to bind to a target structure in vitro or in vivo, for example a cellular receptor. Such a target structure may be inside or, preferably, on the surface of a target cell, and typically inside living multicellular organisms, preferably mammals, most preferably humans. Examples of proteinaceous binding entities are proteins, peptides, or glycoproteins.

A particular binding entity is an antibody or antibody fragment. Such antibodies and antibody fragments with selectivity for a particular target structure are well known in the art. Preferred are recombinant antibody fragments, and preferably humanized antibody fragments when an application in humans is intended. Antibody fragments may, for example be Fab, Fab′ or preferably scFv fragments.

Alternatives to antibodies or antibody fragments known in the art are also considered, for example natural or fully synthetic binder proteins directed to particular receptors within cells or on cell membranes. Particular examples are listed e.g. by Fiedler M. et al. in TRENDS in Biotechnology, 23:514, 2005, and include, but are not limited to, three-helix bundles from Z-domain of Protein A from S. aureus; binders based on human transferrin; monomeric or trimeric human C-type lectin domains; and ankyrin repeat proteins.

Moreover the invention relates to a method of reacting a fusion protein comprising a proteinaceous binding entity directed to a target and an acyl carrier protein (ACP) or a fragment thereof with a coenzyme A (CoA) type compound carrying one or more drug entities and optionally a label detectable by a fluorescence detector, magnetic resonance imaging (MRI), positron emission tomography (PET) or scintigraphy, and a holo-acyl carrier protein synthase (ACPS) or a homologue thereof so that the ACPS transfers the one or more drug entities and the optional detectable label to the fusion protein.

ACP or ACP domains act as carriers in the biosynthesis of fatty acids, polyketides and in non-ribosomal peptide synthesis. ACP is posttranslationally modified by holo-acyl carrier protein synthase (ACPS) which transfers the 4-phosphopantetheine cofactor from the coenzyme A to a conserved serine residue of ACP. ACPSs are also called phosphopantetheinyl transferases. It has been shown that ACPSs possesses a relatively low substrate specificity concerning modifications of CoA at the thiol group of the phosphopantetheinyl moiety of CoA. Taking advantage of this, ACP fusion proteins can be specifically labeled by incubation with ACPS and a CoA derivative that carries the label via the phosphopantetheinyl moiety. The label is thus transferred with the phosphopantetheinyl moiety to the conserved serine residue of the ACP. The labelling is independent of the nature of the fusion protein.

The terms ACP and ACPS here stand for any pair of proteins in which one of the two (the ACP) is an acceptor for a phosphopantetheinyl derivative that originates from a CoA derivative and the other one (the ACPS) catalyzes the transfer of the phosphopantetheinyl derivative to this ACP. The terms ACPS and phosphopantetheinyl transferase are used interchangeably. Examples for further useful phosphopantetheinyl transferases are: EntD, participating in enterobactin synthesis; Sfp and Psf-1, participating in surfactin biosynthesis; Gsp, participating in gramidicin S biosynthesis; LYS5, participating in lysine biosynthesis; Bli, participating in bacitracin biosynthesis; Lpa-14, participating in iturin A biosynthesis; and NshC, participating in nosiheptide biosynthesis. This invention includes the use of all ACPSs that are homologous to the ACPS from E. coli in the described drug transfer.

The term ACP stands for any protein that will be posttranslationally phosphopantetheinylated by ACPS from E. coli or any ACPS homologous to the ACPS from E. coli. This includes proteins not only participating in fatty acid synthesis but also in polyketide synthesis, non-ribosomal peptide synthesis, amino acid synthesis and depsipeptide synthesis. In addition to the posttranslational modification by ACPS these proteins have also in common that they form acyl-pantetheinyl thiolesters with different substrates and that the phosphopantetheinyl moiety is attached to a serine residue. In their natural function the ACPs might be the domain of a multi-functional enzyme (as in type I fatty acid synthases) or a separate protein (as in type II fatty acid synthases).

In a preferred application, the acyl carrier protein or “ACP” has the property of being modified by the holo-acyl carrier protein synthase or “ACPS” in a way that labeled 4′-phosphopantetheine is transferred from the appropriate coenzyme A (“CoA”) to a serine residue of ACP or a fragment thereof forming part of the fusion protein. In preferred embodiments, ACP is, for example, E. coli acyl carrier protein. However, other acyl carrier proteins (ACP) are known and may be used in the invention, e.g. ACP from Streptomyces species, or any ACP having the property of being modified by ACPS defined above in the presence of the CoA compound of the invention. In the present invention, ACP also includes variants of a wild-type ACP which may differ by virtue of one or more amino acid substitutions, deletions or additions, but which still retain the property of serving as an acceptor for labeled 4′-phosphopantetheine in the reaction catalyzed by ACPS. Other variants of ACP may be chemically modified using techniques well known to those skilled in the art. ACP variants may be produced using protein engineering techniques known to the skilled person and/or using molecular evolution to generate and select new acceptor sequences for transfer of labeled 4′-phosphopantetheine in the reaction catalyzed by ACPS. ACP fragments are those which contain the serine residue to which the phosphopantetheine derivative is attached, and which retain the function to accept such phosphopantetheine derivative.

In preferred embodiments, the ACPS is, for example, E. coli holo-acyl carrier protein synthase. However, other holo-acyl carrier protein synthases are known, such as ACPS from Bacillus subtilis. ACPSs are also known as phosphopantetheinyl transferases, and the present invention includes the use of this general class of enzymes. In the present invention, holo-acyl carrier protein synthases also includes variants of a wild-type ACPS which may differ by virtue of one or more amino acid substitutions, deletions or additions, but which still retain the property of transferring labeled 4′-phosphopantetheine specifically to the ACP fusion protein. Other variants of ACPS may be chemically modified using techniques well known to those skilled in the art. ACPS variants may be produced using protein engineering techniques known to the skilled person and/or using molecular evolution to generate and select new specificities for transfer of labeled 4′-phosphopantetheine to different acceptor sequences.

Furthermore the invention relates to pharmaceutical compositions comprising the molecular shuttles as defined hereinbefore, wherein at least one of the cargo entities is a drug, and to a method of cancer treatment comprising administering a molecular shuttle or a pharmaceutical composition comprising a molecular shuttle as defined hereinbefore, wherein at least one of the cargo entities is a drug useful in the treatment of cancer.

The prime application for pharmaceutical compositions of the invention is cancer where molecular shuttles will be administered to inhibit the growth of or to kill selectively cancer cells exhibiting a particular surface structure and showing abnormal growth. Further applications are in the prevention of the growth of harmful structures including one or several particular cell types without neoplastic characteristics, like in atherosclerotic processes, leading to stenosis of blood vessels.

### EXAMPLES

### Abbreviations

CoA-SH=coenzyme A
DIPEA=diisopropylethylamine
DMF=dimethylformamide
DMSO=dimethyl sulfoxide
DTT=dithiothreitol
eq=equivalent
ESI-MS=electrospray ionization mass spectrometry
Et₃N=triethylamine
EtOAc=ethyl acetate
EtOH=ethanol
Fmoc=9-fluorenylmethyoxycarbonyl
HOBT=1-hydroxybenzotriazole
HPLC=high pressure liquid chromatography
Lys=lysine
MeNH₂=methylamine
MeOH=methanol
NHS=N-hydroxy succinimide

### NMP=N-methylpyrrolidine

PBS=phosphate buffered saline
PEG=polyethylene glycol

### PEG12=—(CH₂CH₂O)₁₂—

PMe₃=trimethylphosphine
PYBOP=(benzotriazol-1-yloxy)-tripyrrolidino-phosphonium hexafluorophosphate
rt=room temperature
siRNA=short interfering ribonucleic acid
TFA=trifluoroacetic acid
Tris=tris(hydroxymethyl)methylamine

### Example 1

### Azido-PEG12-propionic acid 2-maleimidoethylamide (1)

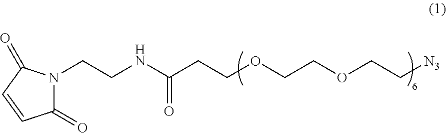

N-(2-aminoethyl)maleimide trifluoroacetate (343 mg, 1.35 mmol) and azido-PEG12-propionic NHS ester (1 g, 1.35 mmol) are dissolved in 5 mL DMF with Et₃N (188 μL, 1.35 mmol) and heated overnight at 31° C. The solvent is evaporated under vacuum and the product is isolated by reversed phase HPLC on a C18 column using a linear gradient of water:acetonitrile (from 95:5 to 20:80 in 20 min, 0.08% TFA).

ESI-MS: m/z 766.40 [M+H]⁺.

### Example 2

### Azido-PEG12-propionic acid 2-(CoA-S-succinimido)ethylamide (2)

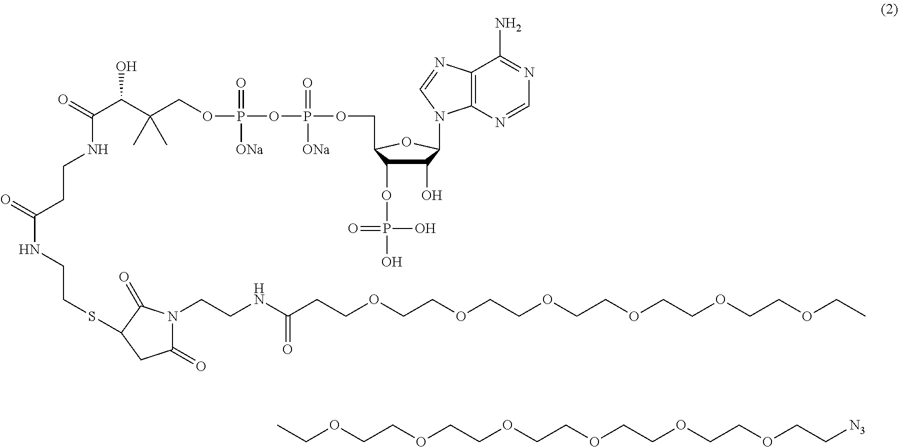

A solution of maleimide derivative (1) (192 mg, 1 eq, 252 μmol) in DMF (2 mL) is added to a solution of CoA-SH (248 mg, 1.2 eq, 304 μmol) in Tris-buffer (pH 7.5, 200 μL). The reaction mixture is shaken overnight at 31° C. The solvent is removed under vacuum and the crude mixture is purified via preparative HPLC. ESI-MS: m/z 1554.48 [M-Na]⁻.

### Example 3

### Amino-PEG12-propionic acid 2-(CoA-S-succinimido)ethylamide (3)

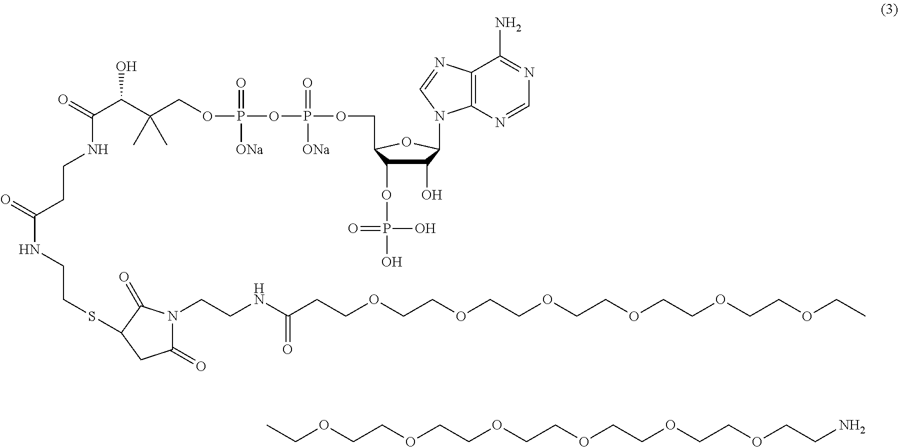

To a solution of compound (2) (204 mg, 0.13 mmol) in dioxane (3 mL) water (450 μL) is added. PMe₃ (800 μl_{—} 1 M in THF, 6 eq) is added and the solution is stirred at rt for 2 h. The solvent is removed under reduced pressure, and compound (3) is obtained by purification with preparative HPLC. ESI-MS: m/z 1527.48 [M-Na]⁻.

### Example 4

### Chlorambucil-amino-PEG12-propionic acid 2-(CoA-S-succinimido)ethylamide (4)

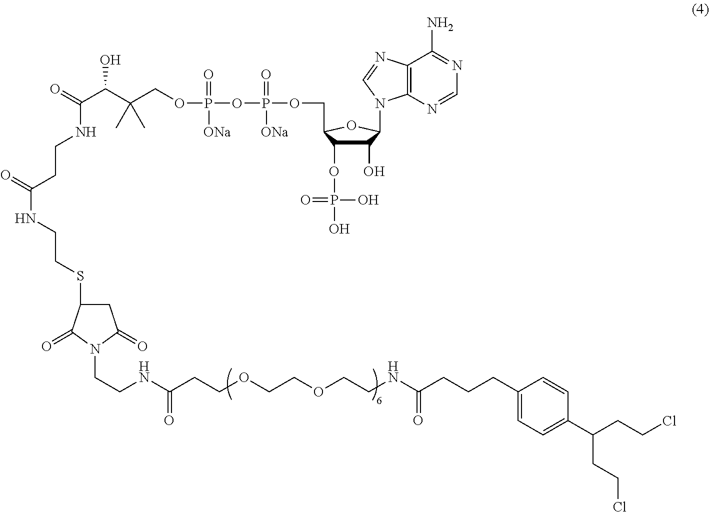

To a solution of chlorambucil (1.2 mg, 0.004 mmol) in DMF (1 mL) PYBOP (2 mg, 0.004 mmol) is added at rt. The solution is stirred at rt for 20 min. Compound (3) (6.1 mg, 0.004 mmol) and DIPEA (0.6 μL, 0.004 mmol) are added and the solution is heated at 50° C. for 5 min. The solution is stirred at rt overnight. The solvent is removed under reduced pressure. The compound is isolated by reversed phase HPLC on a C18 column using a linear gradient of water:acetonitrile (from 95:5 to 20:80 in 20 min, 0.08% TFA).

ESI-MS: m/z 1813.58 [M-Na]⁻.

### Example 5

### 6-Maleimido-hexanoylamino-PEG12-propionic acid 2-(CoA-S-succinimido)-ethylamide (5)

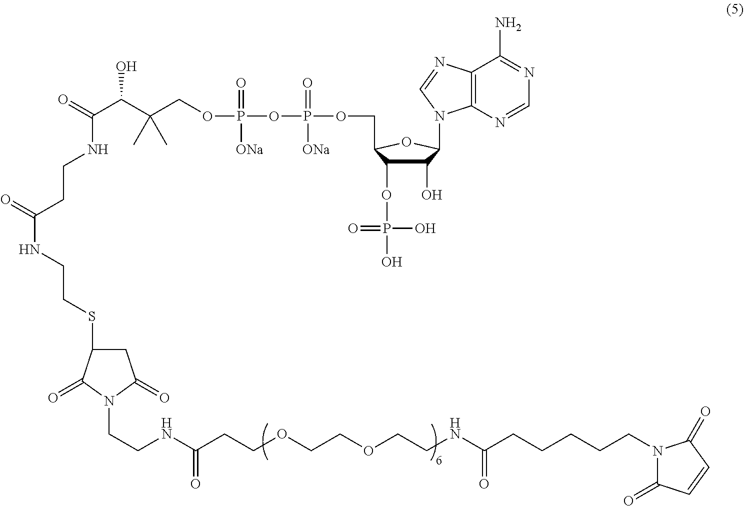

To a solution of 6-maleimido-hexanoic acid (2 mg, 0.009 mmol) in DMF (1 mL) PYBOP (5 mg, 0.009 mmol) is added at rt. The solution is stirred at rt for 20 min. Compound (4) (14 mg, 0.009 mmol) and DIPEA (1.5 μL, 0.009 mmol) are added and the solution is heated at 50° C. for 5 min. The solution is stirred at rt overnight. The solvent is removed under reduced pressure. The compound is isolated by reversed phase HPLC on a C18 column using a linear gradient of water:acetonitrile (from 95:5 to 20:80 in 20 min, 0.08% TFA). ESI-MS: m/z 1721.57 [M-Na]⁻.

### Example 6

### 6-Maleimido-hexanoylamino-PEG12-propionic acid 2-(CoA-S-succinimido)-ethylamide siRNA conjugate (6)

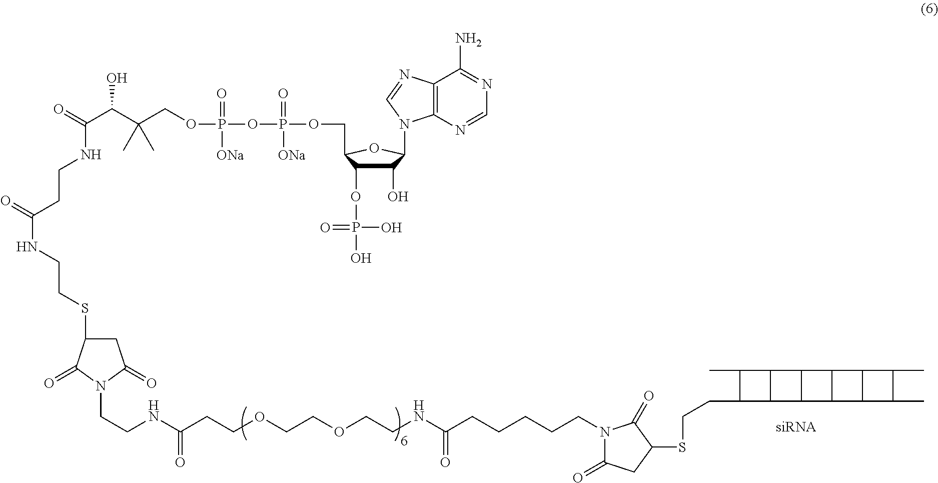

The 5′-thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL. 300 μL of a solution of compound (5) (2.5 mM in DMF) is added and the reaction mixture incubated at rt for 1 h. The reaction mixture is diluted with water to a total volume of 2 mL and excess maleimide removed by gel filtration. The siRNA conjugate (6) is then purified by HPLC (solvent A: 0.1 M tetraethylammonium acetate pH 6.9 in water; solvent B: acetonitrile).

### Example 7

### 5-Fluorescein-Lys-Fmoc-OH (7) and 6-fluorescein-Lys-Fmoc-OH (8)

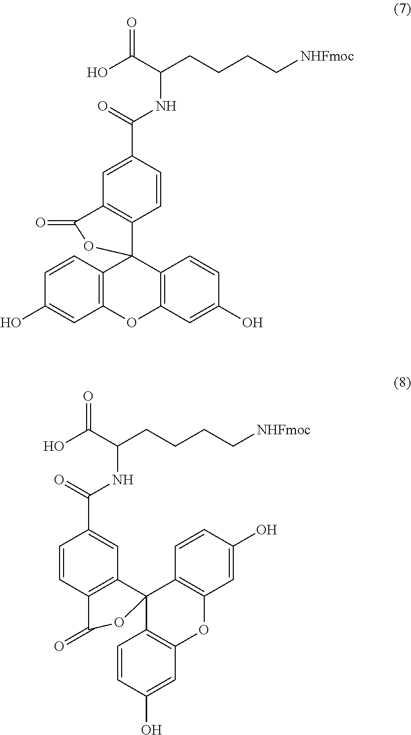

Fmoc-Lys-OH (184 mg, 0.5 mmol) and 5(6)-carboxyfluorescein NHS ester (237 mg, 0.5 mmol) are dissolved in 5 mL of DMF with Et₃N (70 μL, 0.5 mmol) and heated overnight at 31° C. The crude mixture is poured onto water (100 mL). The aqueous solution is basified (pH=9) with NaOH (1 M). The aqueous phase is washed with ethyl acetate. Upon acidification of the aqueous phase with acetic acid, a yellowish precipitate is formed. The solid is collected via filtration to afford the desired compound as a mixture of isomers (7) and (8). ESI-MS: m/z 727.7 [M+H]⁺.

### Example 8

### 5-Fluorescein-Lys-OH (9) and 6-fluorescein-Lys-OH (10)

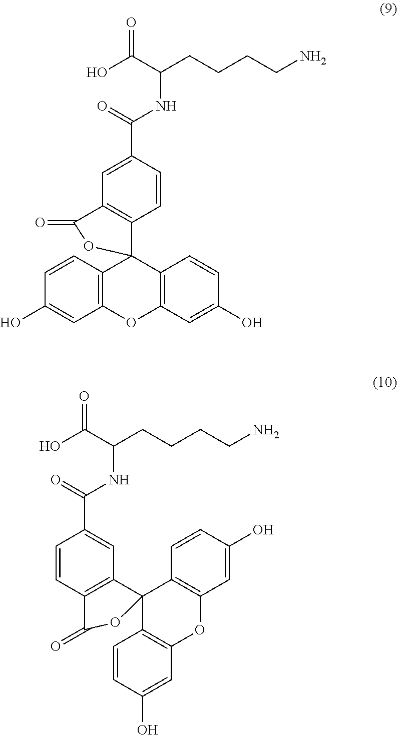

To a solution of mixture of compounds (7) and (8) (300 mg, 0.4 mmol) in DMF (3 mL) diethylamine (600 μL) is added at rt. The solution is stirred at rt for 3 h. The solvent is removed under reduced pressure and the desired mixture of compounds (9) and (10) is directly used for the next step. ESI-MS: m/z 505.15 [M+H]⁺.

### Example 9

### N-5-Fluorescein-N′-chlorambucil-Lys-OH (11) and N-6-fluorescein-N′-chlorambucil-Lys-OH (12)

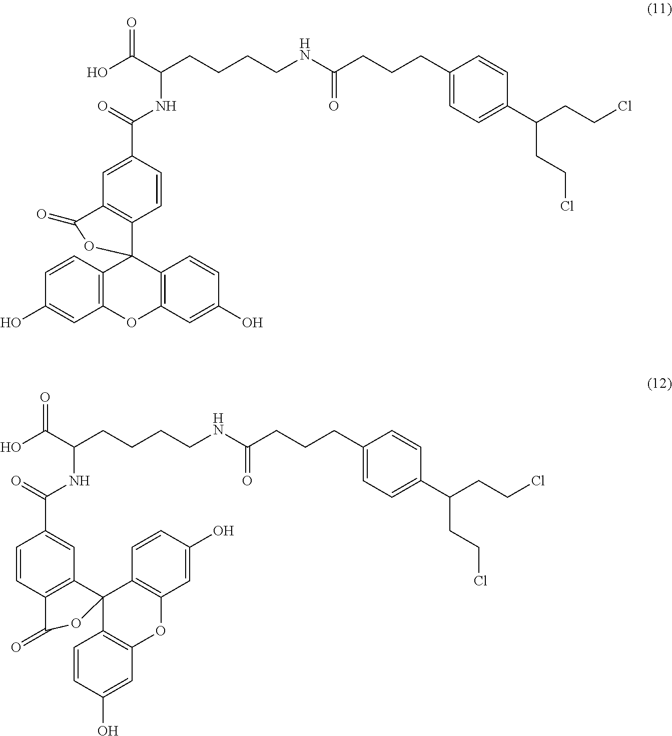

To a solution of chlorambucil (106 mg, 0.35 mmol) in DMF (3 mL) PYBOP (182 mg, 0.35 mmol) is added at rt. The solution is stirred at rt for 20 min. The mixture of isomers (9) and (10) (176 mg, 0.35 mmol) and DIPEA (58 μL, 0.35 mmol) are added and the solution is heated at 50° C. for 5 min. The solution is stirred at rt overnight. The crude mixture is poured into water (60 mL). The aqueous solution is basified (pH=9) with NaOH (1 M). The aqueous phase is washed with ethyl acetate. Upon acidification of the aqueous phase with acetic acid, a yellowish precipitate is formed. The solid is collected via filtration to afford the desired compound as a mixture of isomers (11) and (12).

ESI-MS: m/z 789.23 [M+H]⁺.

### Example 10

### N′-5-Fluorescein-N″-chlorambucil-Lys-amido-PEG12-propionic acid 2-(CoA-S-succinimido)ethylamide (13) and corresponding 6-fluorescein derivative (14)

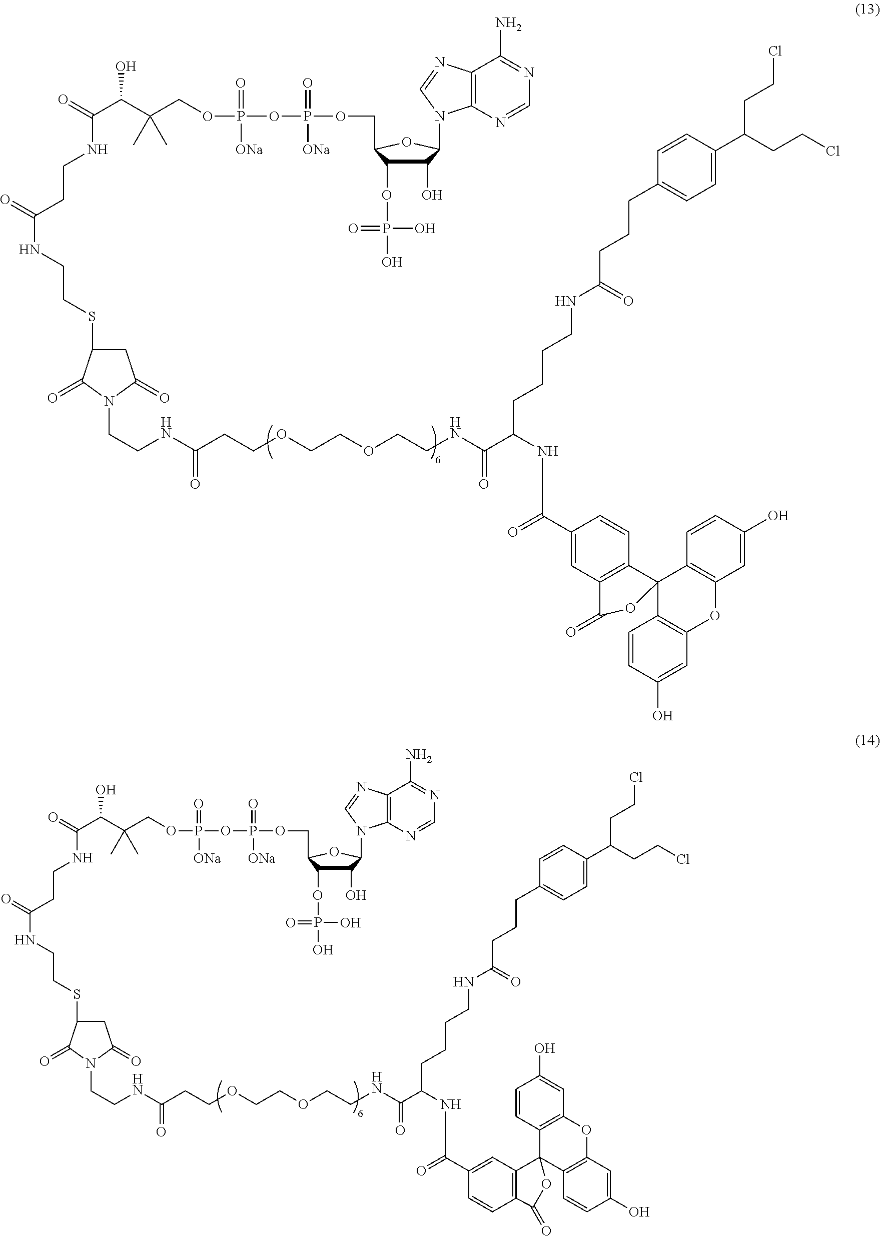

To a solution of a mixture of isomers (11) and (12) (24 mg, 0.03 mmol) in DMF (3 mL) PYBOP (16 mg, 0.03 mmol) is added at rt. The solution is stirred at rt for 20 min. Amino-PEG12-propionic acid 2-(CoA-S-succinimido)ethylamide (3) (46 mg, 0.03 mmol) and DIPEA (5 μL, 0.03 mmol) are added and the solution is heated at 50° C. for 5 min. The solution is stirred at rt overnight. The solvent is removed under reduced pressure, and compounds (13) and (14) are obtained by purification with preparative HPLC.

ESI-MS: m/z 2300.05 [M-Na]⁻.

### Example 11

### N-5-Fluorescein-N′-6-maleimidohexanoyl-Lys-OH (15) and N-6-fluorescein-N′-6-maleimidohexanoyl-Lys-OH (16)

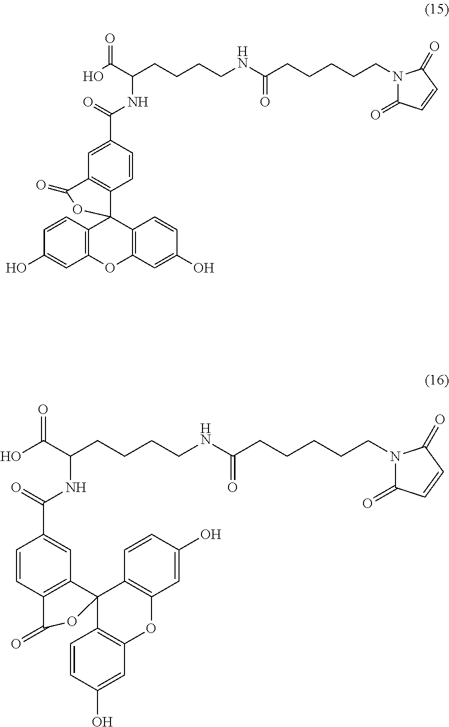

To a solution of 6-maleimido-hexanoic acid (66 mg, 0.31 mmol) in DMF (3 mL) PYBOP (161 mg, 0.31 mmol) is added at rt. The solution is stirred at rt for 20 min. The mixture of compounds (9) and (10) (156 mg, 0.31 mmol) and DIPEA (51 μL, 0.31 mmol) are added and the solution is heated at 50° C. for 5 min. The solution is stirred at rt overnight. The crude mixture is poured into water (60 mL). The aqueous solution is basified (pH=9) with NaOH (1 M). The aqueous phase is washed with ethyl acetate. Upon acidification of the aqueous phase with acetic acid, a yellowish precipitate is formed. The solid is collected via filtration to afford the desired compound as a mixture of isomers (15) and (16).

ESI-MS: m/z 699.23 [M+H]⁺.

### Example 12

### N′-5-Fluorescein-N″-6-maleimidohexanoyl-Lys-amido-PEG12-propionic acid 2-(CoA-S-succinimido)ethylamide (17) and corresponding 6-fluorescein derivative (18)

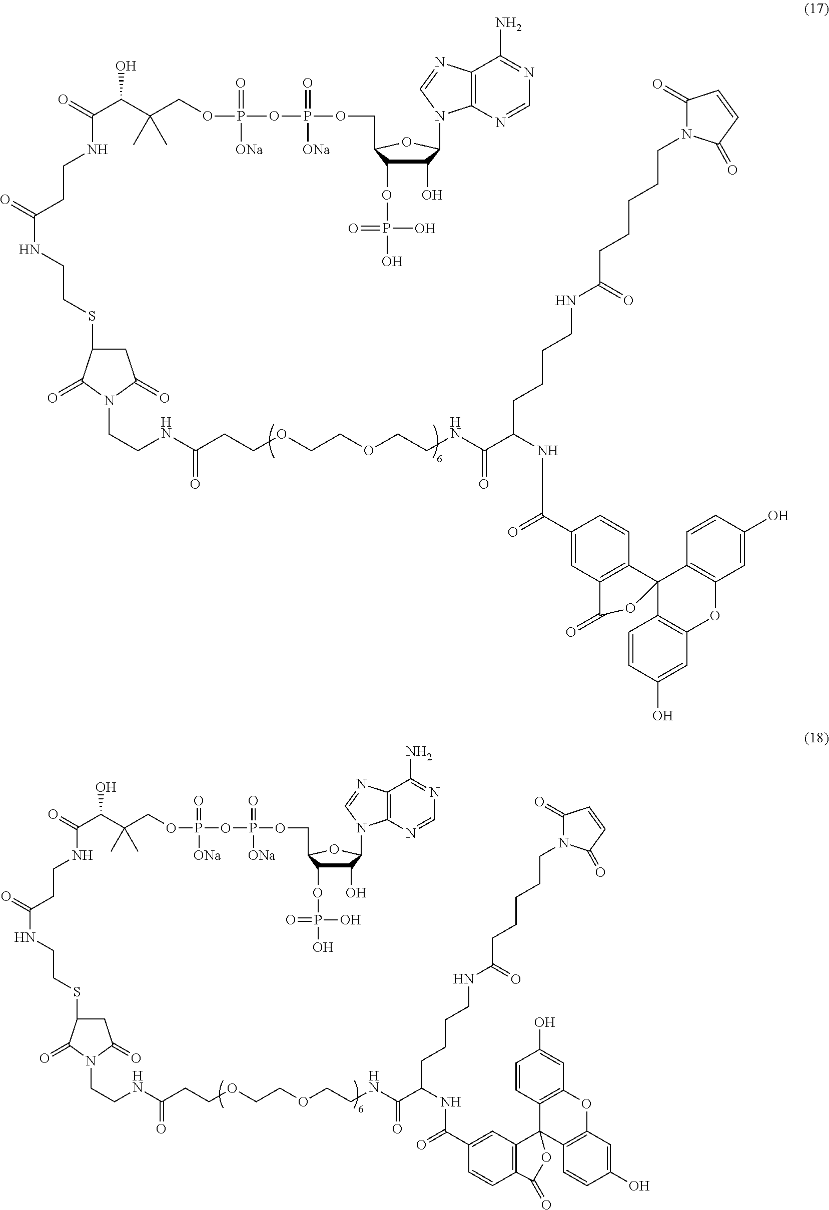

To a solution of a mixture of isomers (15) and (16) (14 mg, 0.02 mmol) in DMF (2 mL) PYBOP (10 mg, 0.02 mmol) is added at rt. The solution is stirred at rt for 20 min. Amino-PEG12-propionic acid 2-(CoA-S-succinimido)ethylamide (3) (31 mg, 0.02 mmol) and DIPEA (3.3 μL, 0.02 mmol) are added and the solution is heated at 50° C. for 5 min. The solution is stirred at rt overnight. The solvent is removed under reduced pressure, and compounds (17) and (18) are obtained by purification with preparative HPLC.

ESI-MS: m/z 2208.04 [M-Na]⁻.

### Example 13

### N′-5-Fluorescein-N″-6-maleimidohexanoyl-Lys-amido-PEG12-propionic acid 2-(CoA-S-succinimido)ethylamide siRNA conjugate (19) and corresponding 6-fluorescein derivative (20)

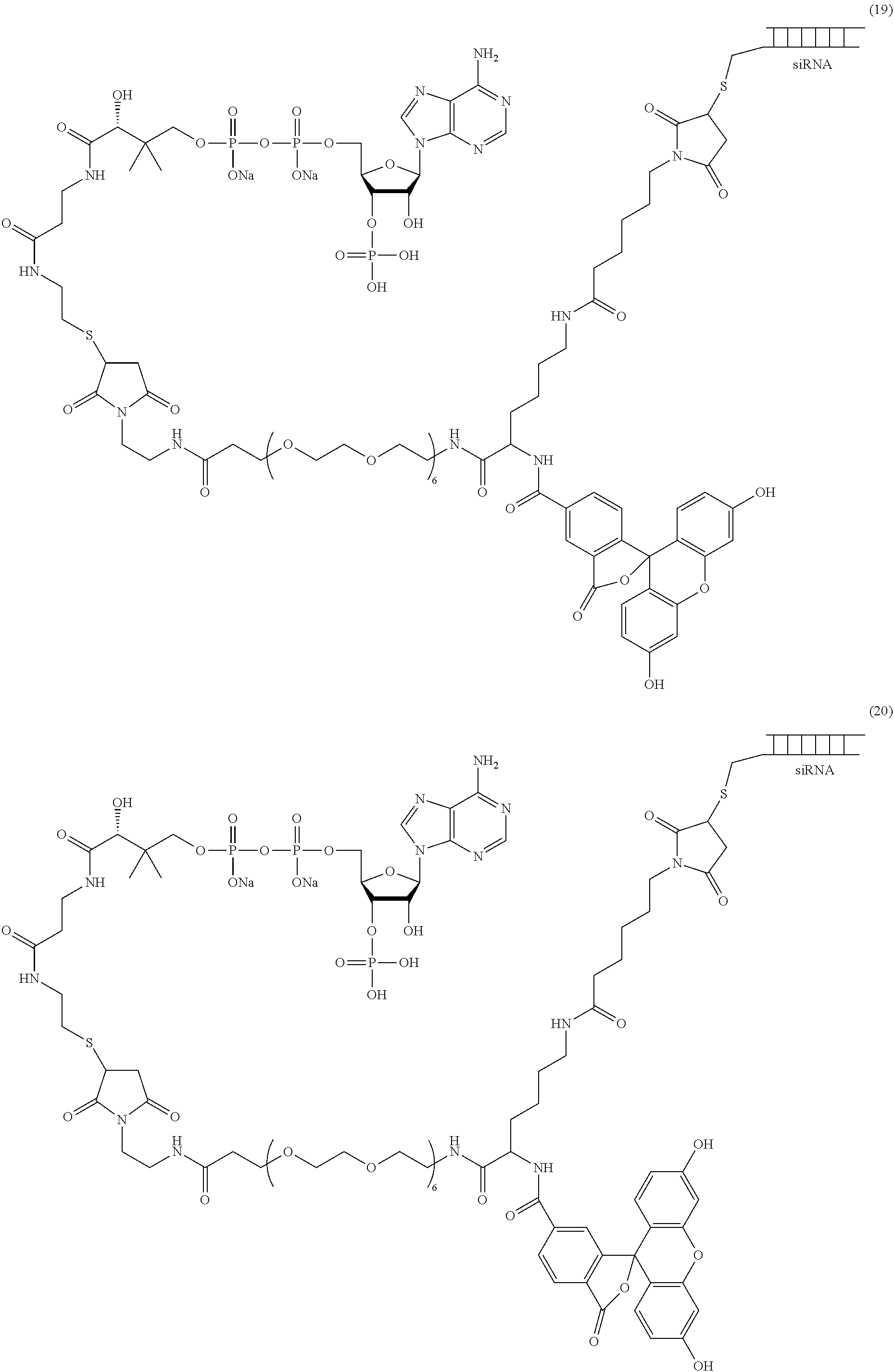

The 5′-thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL. 300 μL solution of a mixture of compounds (17) and (18) (2.5 mM in DMF) is added and the reaction mixture incubated at room temperature for 1 h. The reaction mixture is diluted with water to a total volume of 2 mL and excess maleimide removed by gel filtration. The conjugates (19) and (20) are then purified by HPLC (solvent A: 0.1 M tetraethylammonium acetate pH 6.9 in water; solvent B: acetonitrile).

### Example 14

### N,N′-Di(chlorambucil)-Lys-OH (21)

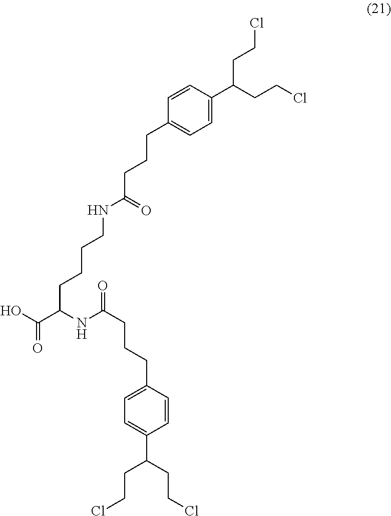

To a solution of chlorambucil (106 mg, 0.70 mmol) in DMF (3 mL) PYBOP (182 mg, 0.70 mmol) is added at rt. The solution is stirred at rt for 20 min. Lysine (51 mg, 0.35 mmol) and DIPEA (116 μL, 0.70 mmol) are added and the solution is heated at 50° C. for 5 min. The solution is stirred at rt overnight. Then the crude mixture is poured into water (60 mL). The aqueous phase is basified (pH=9) with NaOH (1 M). The aqueous phase is washed with ethyl acetate. Upon acidification of the aqueous phase with acetic acid, a yellowish precipitate is formed. The solid is collected via filtration to afford compound (21).

ESI-MS: m/z 717.21 [M+H]⁺.

### Example 15

### N,N′-Di(chlorambucil)-Lys-amido-PEG12-propionic acid 2-(CoA-S-succinimido)ethylamide (22)

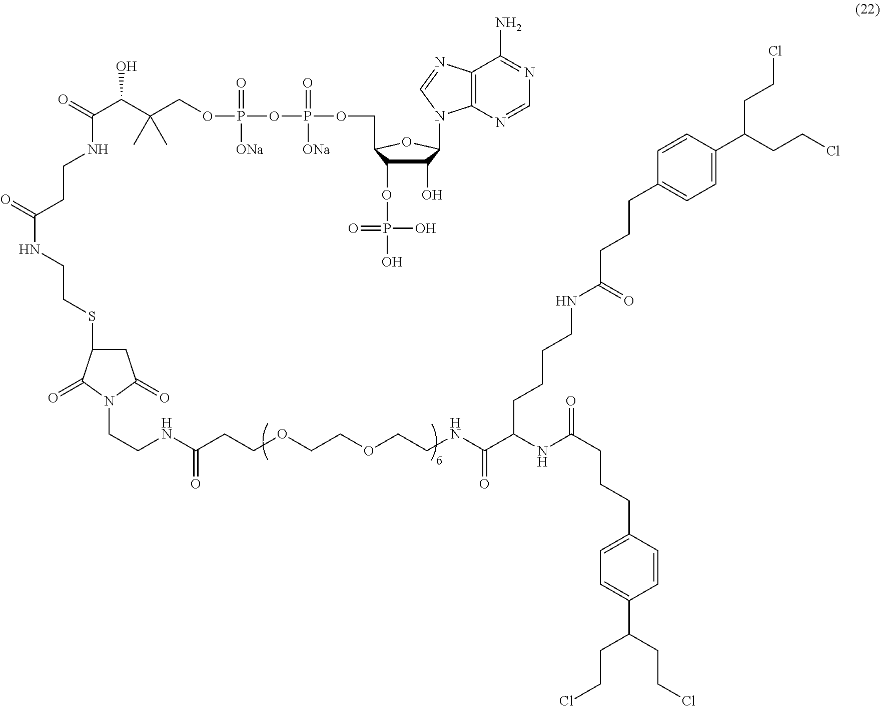

To a solution of (21) (15 mg, 0.022 mmol) in DMF (2 mL) PYBOP (11 mg, 0.022 mmol) is added at rt. The solution is stirred at rt for 20 min. Compound (3) (34 mg, 0.022 mmol) and DIPEA (3.6 μL, 0.022 mmol) are added and the solution is heated at 50° C. for 5 min. The solution is stirred at rt overnight. The solvent is removed under reduced pressure, and compound (22) is obtained by purification with preparative HPLC.

ESI-MS: m/z 2250.9 [M-Na]⁻.

### Example 16

### N,N′-Di(6-maleimidohexanoyl)-Lys-OH (23)

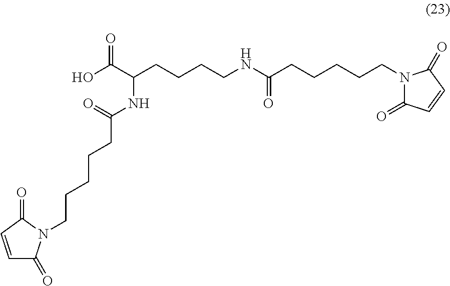

To a solution of 6-maleimido-hexanoic acid (126 mg, 0.6 mmol) in DMF (3 mL) PYBOP (156 mg, 0.6 mmol) is added at rt. The solution is stirred at rt for 20 min. Lysine (44 mg, 0.3 mmol) and DIPEA (100 μL, 0.6 mmol) are added and the solution is heated at 50° C. for 5 min. The solution is stirred at rt overnight. The crude mixture is poured into water (60 mL). The aqueous phase is basified (pH=9) with NaOH (1 M). The aqueous phase is washed with ethyl acetate. Upon acidification of the aqueous phase with acetic acid, a yellowish precipitate is formed. The solid is collected via filtration to afford compound (23).

ESI-MS: m/z 533.59 [M+H]⁺.

### Example 17

### N,N′-Di(6-maleimidohexanoyl)-Lys-amido-PEG12-propionic acid 2-(CoA-S-succinimido)ethylamide (24)

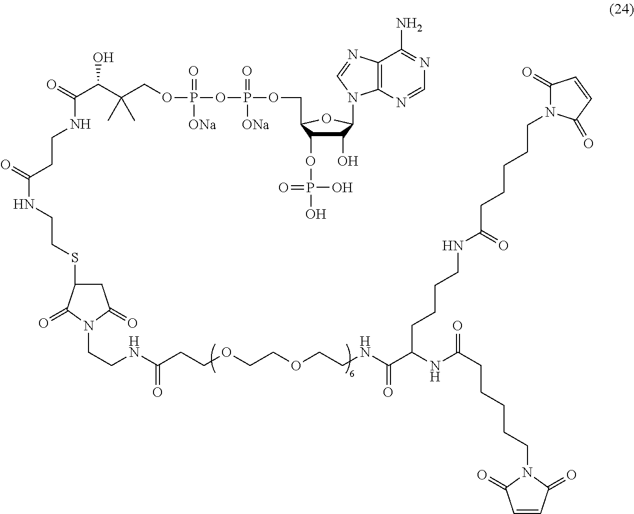

To a solution of compound (23) (9 mg, 0.017 mmol) in DMF (2 mL) PYBOP (9 mg, 0.017 mmol) is added at rt. The solution is stirred at rt for 20 min. Compound (3) (26 mg, 0.017 mmol) and DIPEA (2.8 μL, 0.017 mmol) are added and the solution is heated at 50° C. for 5 min. The solution is stirred at rt overnight. The solvent is removed under reduced pressure, and compound (24) is obtained by purification with preparative HPLC.

ESI-MS: m/z 2042.94 [M-Na]⁻.

### Example 18

### N,N′-Di(6-siRNA-sucinimidohexanoyl)-Lys-amido-PEG12-propionic acid 2-(CoA-S-succinimido)ethylamide (25)

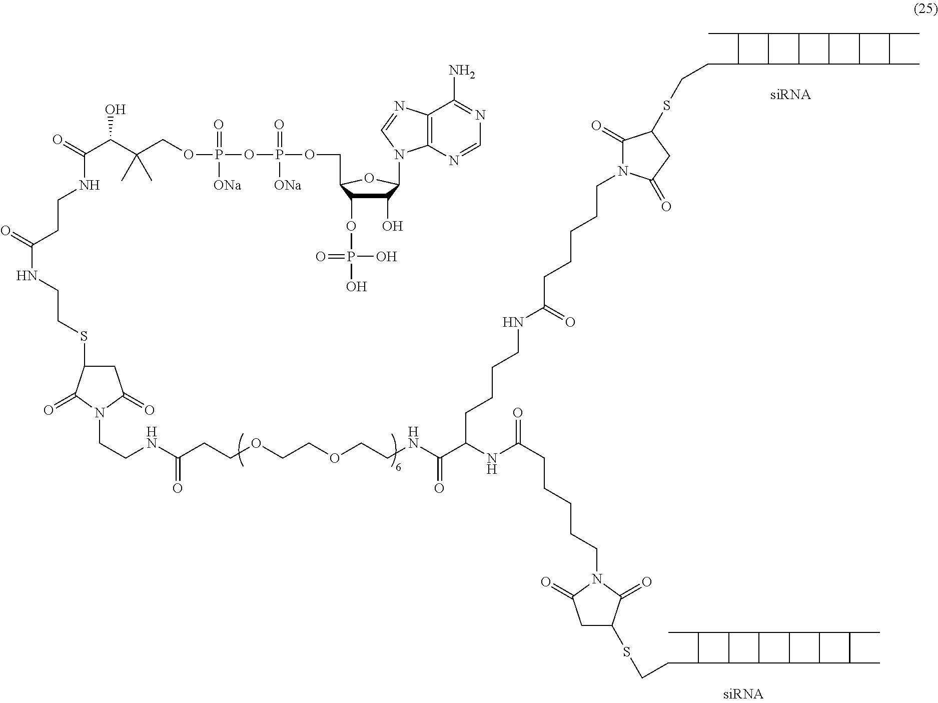

The 5′-thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL. 300 μL solution of compound (24) (2.5 mM in DMF) is added and the reaction mixture incubated at room temperature for 1 h. The reaction mixture is diluted with water to a total volume of 2 mL and excess maleimide removed by gel filtration. The conjugate (25) is then purified by HPLC (solvent A: 0.1 M tetraethylammonium acetate pH 6.9 in water; solvent B: acetonitrile).

### Example 19

### N-(6-Maleimidohexanoyl)-N′-Fmoc-Lys-OH (26)

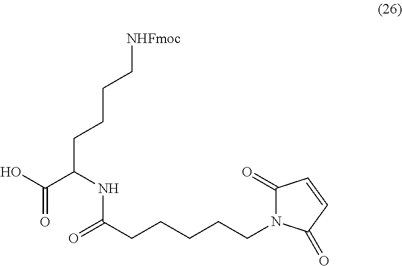

To a solution of 6-maleimido-hexanoic acid (88 mg, 0.42 mmol) in DMF (3 mL) PYBOP (109 mg, 0.42 mmol) is added at rt. The solution is stirred at rt for 20 min. Fmoc-Lys-OH (155 mg, 0.42 mmol) and DIPEA (70 μL, 0.42 mmol) are added and the solution is heated at 50° C. for 5 min. The solution is stirred at rt overnight. The crude mixture is poured into water (60 mL). The aqueous phase is basified (pH=9) with NaOH (1 M). The aqueous phase is washed with ethyl acetate. Upon acidification of the aqueous phase with acetic acid, a yellowish precipitate is formed. The solid is collected via filtration to afford compound (26). ESI-MS: m/z 562.63 [M+H]⁺.

### Example 20

### 6-Maleimidohexanoyl-Lys-OH (27)

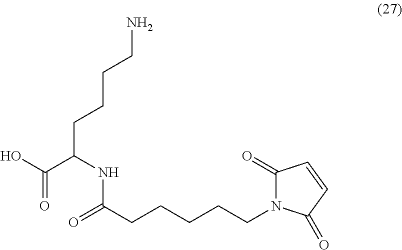

To a solution of compound (26) (163 mg, 0.29 mmol) in DMF (3 mL) diethylamine (600 μL) is added at rt. The solution is stirred at rt for 3 h. The solvent is removed under reduced pressure and the desired compound (27) is directly used for the next step.

ESI-MS: m/z 340.39 [M+H]⁺.

### Example 21

### N-6-Maleimidohexanoyl-N′-chlorambucil-Lys-OH (28)

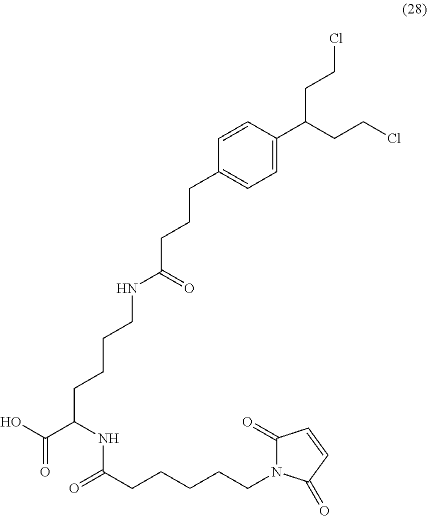

To a solution of chlorambucil (53 mg, 0.17 mmol) in DMF (2 mL) PYBOP (91 mg, 0.17 mmol) is added at rt. The solution is stirred at rt for 20 min. Compound (27) (58 mg, 0.17 mmol) and DIPEA (29 μL, 0.17 mmol) are added and the solution is heated at 50° C. for 5 min. The solution is stirred at rt overnight. The crude mixture is poured into water (40 mL). The aqueous solution is basified (pH=9) with NaOH (1 M). The aqueous phase is washed with ethyl acetate. Upon acidification of the aqueous phase with acetic acid, a yellowish precipitate is formed. The solid is collected via filtration to afford the desired compound (28). ESI-MS: m/z 625.6 [M+H]⁺.

### Example 22

### N-6-Maleimidohexanoyl-N′-chlorambucil-Lys-amido-PEG12-propionic acid 2-(CoA-S-succinimido)ethylamide (29)

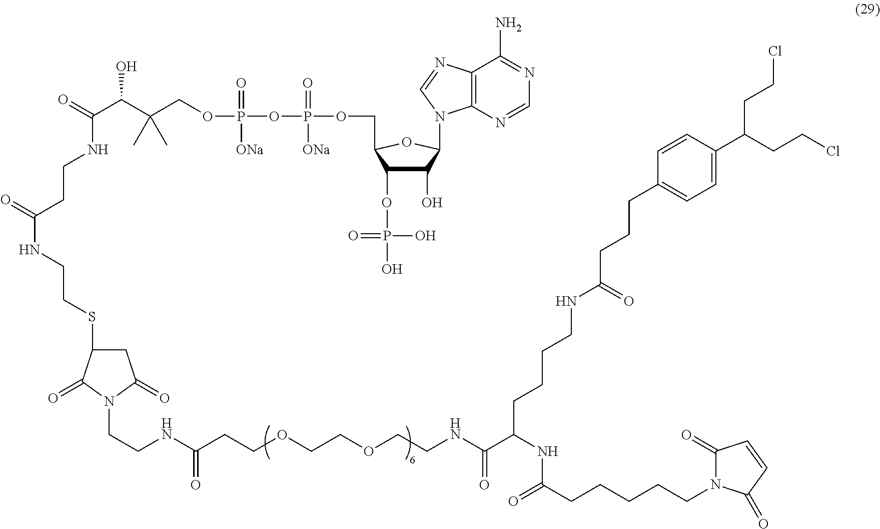

To a solution of compound (28) (13 mg, 0.021 mmol) in DMF (2 mL) PYBOP (11 mg, 0.021 mmol) is added at rt. The solution is stirred at rt for 20 min. Compound (3) (32 mg, 0.021 mmol) and DIPEA (3.5 μL, 0.021 mmol) are added and the solution is heated at 50° C. for 5 min. The solution is stirred at rt overnight. The solvent is removed under reduced pressure, and compound (29) is obtained by purification with preparative HPLC.

ESI-MS: m/z 2134.93 [M-Na]⁻.

### Example 23

### N-6-Maleimidohexanoyl-N′-chlorambucil-Lys-amido-PEG12-propionic acid 2-(CoA-S-succinimido)ethylamide siRNA conjugate (30)

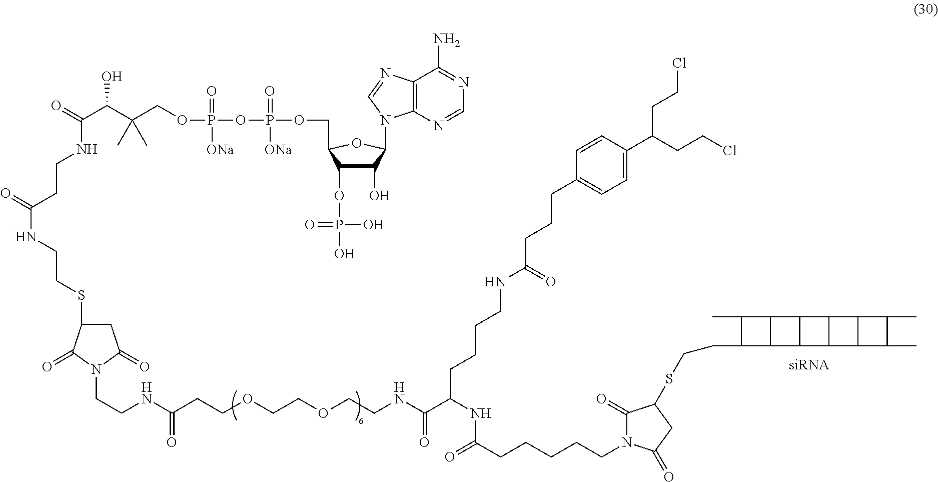

The 5′-thiol modified oligonucleotide (43 nmol) is reduced by incubation for 1 h at rt with 200 mM DTT in 200 μL Tris-buffer pH 8.5. DTT is removed by gel filtration and the oligonucleotide eluted in PBS (pH 7.4). The most concentrated fractions are combined giving a total of 800 μL. 300 μL solution of compound (29) (2.5 mM in DMF) is added and the reaction mixture incubated at room temperature for 1 h. The reaction mixture is diluted with water to a total volume of 2 mL and excess maleimide removed by gel filtration. The siRNA conjugate (30) is purified by HPLC (solvent A: 0.1 M tetraethylammonium acetate pH 6.9 in water; solvent B: acetonitrile).

### Example 24

### Fusion Protein Comprising ACP and Cargo Consisting of Chlorambucil

The purified compound (4) of Example 4 comprising the CoA-based substrate linked to chlorambucil is mixed in PBS, pH 7.4, containing 1 mM DTT, at a concentration of 15 μM with 0.66 times this concentration of the fusion protein ACP-FKBP (plasmid for FKBP available from Ariad Pharmaceuticals, USA) and 0.15 times this concentration of ACPS from E. coli as available from Covalys AG, Witterswil as the synthase enzyme. The mixture is reacted for 24 h in the dark. The mixture is separated using gel permeation chromatography. The peak with the highest mass, corresponding to a completely modified shuttle structure is isolated and stored for further use at 4° C. in the dark.

### Example 25

### Fusion Protein Comprising ACP and Cargo Consisting of Chlorambucil and Fluorescein

The purified mixture of compounds (13) and (14) of Example 10 comprising the CoA-based substrate linked to both chlorambucil and fluorescein is mixed in PBS, pH 7.4, containing 1 mM DTT, at a concentration of 15 μM with 0.66 times this concentration of the fusion protein ACP-FKBP and 0.15 times this concentration of ACPS from E. coli as available from Covalys AG, Witterswil as the synthase enzyme. The mixture is reacted for 24 h in the dark. The mixture is separated using gel permeation chromatography. The peak with the highest mass, corresponding to a completely modified shuttle structure is isolated and stored for further use at 4° C. in the dark.

### Example 26

### Tris{[2-(tert-butoxycarbonyl)ethoxy]methyl}methylamine (31)

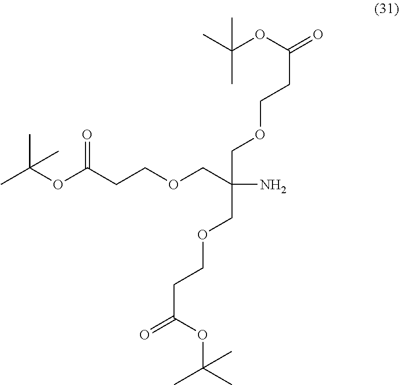

Tris(hydroxymethyl)methylamine (Tris, 2.42 g, 20.0 mmol) in 4.0 mL of a newly opened bottle of DMSO is cooled to 15.0° C. 0.4 mL of 5.0 M NaOH is injected while stirring, followed by tert-butyl acrylate (10.0 mL, 68 mmol), which is injected dropwise. A solvent mixture of 5-10% water in DMSO is optimal for this reaction. The reaction mixture is allowed to reach room temperature and left stirring for 24 h. The crude mixture is poured into water and extracted with ethyl acetate, the organic phase is dried over MgSO₄, and evaporated under reduced pressure to afford compound (31). The compound is directly used for next step without further purification. FAB-MS: m/z 506 [M+H]⁺.

### Example 27

### N-Tris{[2-(tert-butoxycarbonyl)ethoxy]methyl}methyl nile red-oxyacetamide (32)

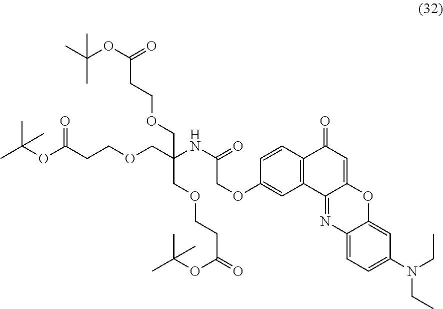

To a solution of nile red-oxyacetic acid (9-diethylamino-5-oxo-benzo[a]phenoxazin-2-oxyacetic acid (100 mg, 0.255 mmol, 1 eq) in DMF (50 mL) are successively added DCC (160 mg, 0.765 mmol, 3 eq) and NHS (90 mg, 0.765 mmol, 3 eq). The resulting mixture is stirred overnight. DCU salts are removed by centrifugation. Compound (31) (130 mg, 0.255 mmol, 1 eq) and DIPEA (42 μL, 0.255 mmol, 1 eq) are added to the solution at rt. The resulting mixture is stirred overnight. The solvent is removed under reduce pressure. Flash chromatography (CH₂Cl₂/methanol, 10/1→5/1) gives the desired compound (32).

MS (ESI) m/z 881 [M+H]⁺.

### Example 28

### N-Tris[(2-carboxyethoxy)methyl]methyl nile red-oxyacetamide (33)

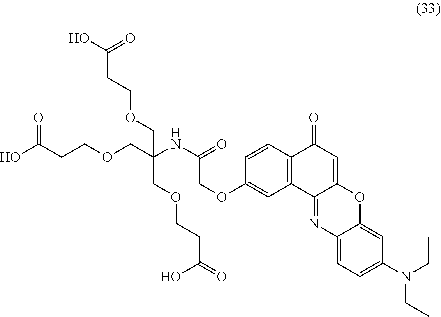

Compound (32) (70 mg, 0.08 mmol) is stirred in 250 μL of 96% formic acid for 18 h. Formic acid is removed under reduced pressure at 50° C. to produce a red oil in quantitative yield. MS (ESI) m/z 712 [M+H]⁺.

### Example 29

### N-Tris-{[2-(N-(2-maleimidoethyl)-aminocarbonyl)ethoxy]methyl}methyl nile red-oxyacetamide (34)

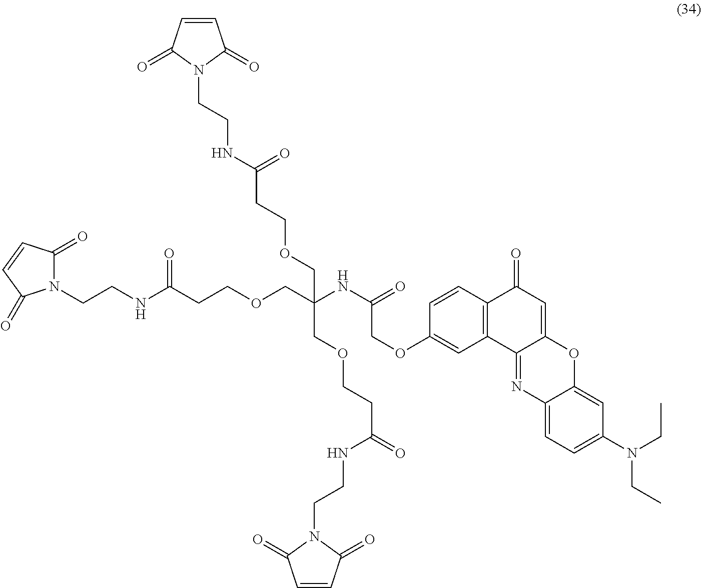

To a solution of N-tris[(2-carboxyethoxy)methyl]methyl nile red-oxyacetamide (33) (10 mg, 0.014 mmol) and N-(2-aminoethyl)maleimide trifluoracetate salt (26 mg, 0.028 mmol, 7 eq) in DMF (1 mL) are successively added pyridine (16 μL, 0.196 mmol, 14 eq), HOBT (1 M in NMP, 14 μL, 0.014 mmol, 1 eq) and EDC (20 mg, 0.098 mmol, 7 eq) at rt. The resulting mixture is stirred overnight. The solvent is evaporated under vacuum and compound (34) isolated by reversed phase HPLC on a C18 column using a linear gradient of water:acetonitrile (from 95:5 to 20:80 in 20 min, 0.08% TFA).

MS (ESI) m/z 1079 [M+H]⁺.

### Example 30

### N-Tris-{[2-(2-(2-(CoA-S)-succinimido)ethylaminocarbonyl)ethoxy]-methyl}methyl nile red-oxyacetamide (35)

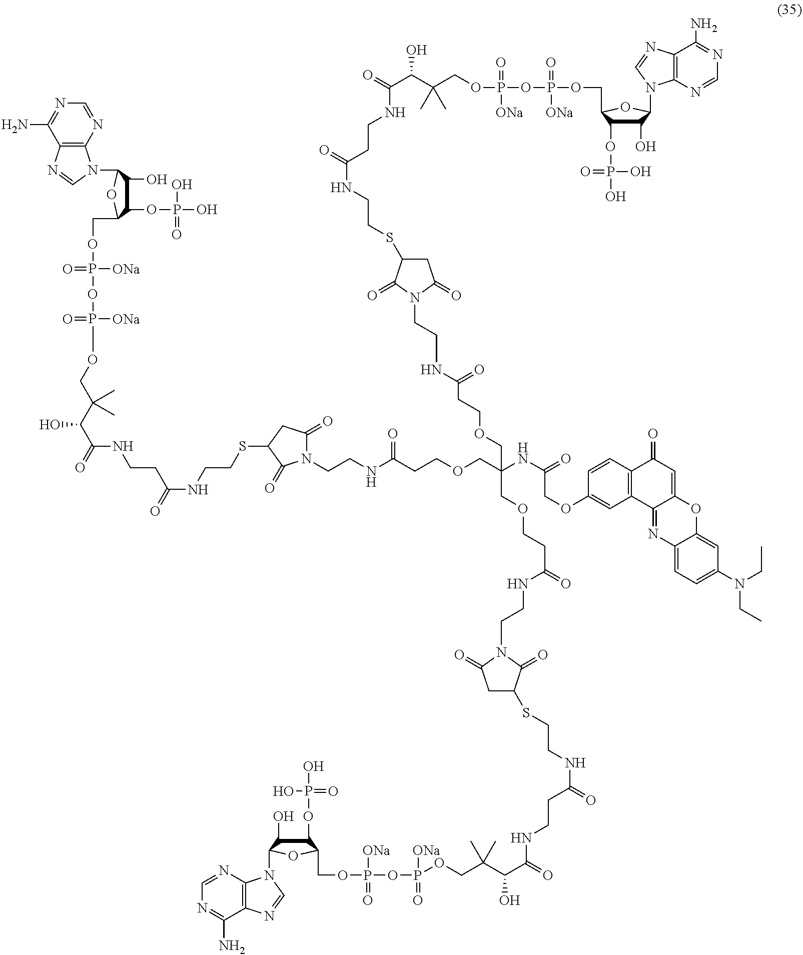

A solution of maleimide derivative (34) (1.4 mg, 1 eq, 1.3 μmol) in DMF (0.7 mL) is added to a solution of CoA-SH (3.5 mg, 3.3 eq, 4.3 μmol) in Tris buffer (pH 7.5, 100 μL). The reaction mixture is shaken overnight at 31° C. The solvent is removed under vacuum and the crude mixture is purified via preparative HPLC. The structural ability of compound (35) to trigger the formation of a protein trimer is confirmed by in vitro experiments using the fusion protein ACP-FRB. 12.5 μL of a 48.5 μM solution of FRB protein fused to the ACP tag (Covalys) is mixed with 0.5 μL of a 0.2 mM solution of compound (35), 2.5 μL of a solution of [500 mM Tris-HCl pH 7.5; 1500 mM NaCl; 100 mM MgCl₂], 9.5 μL of double distilled water and 0.5 μl of ACP synthase enzyme (available from Covalys). Following overnight incubation at 4° C., 5 μL of a solution of 100 mM Tris-HCl pH 6.8; 2% SDS; 35% glycerol; 10 mM EDTA; 20 mM DTT is added. The mixture is boiled for 5 min at 95° C. After cooling to rt, 30 μL of this solution is loaded on a 4-20% linear gradient SDS-PAGE gel. After electrophoresis, the proteins are coomassie stained in gel to visualize protein trimer.

### Example 31

### 3-[2-(2-Maleimidoethyl)disulfanyl]propanoic acid (36)

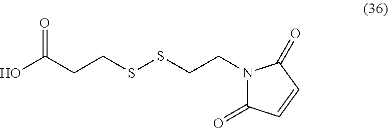

A solution of 3-[2-(2-aminoethyl)disulfanyl]propanoic acid (250 mg, 1.38 mmol) and maleic anhydride (272 mg, 2.76 mmol) in a mixture of acetic acid/toluene (3/1, 3 mL) is heated overnight at 120° C. The crude mixture is cooled down to rt, and further cooled in an ice bath to 0° C. Pentane (50 mL) is added, and a precipitate is formed. Diethyl ether is added to this precipitate, and the white solid formed is removed. The ether solution is concentrated under vacuum to yield the product (36). No further purification is required. ¹H NMR ((CD₃)₂SO, 400 MHz): 7.4 (s, 1H), 6.7 (s, 2H), 3.7 (m, 2H), 2.9 (m, 4H), 2.6 (m, 2H).

### Example 32

### N-Tris{[2-(tert-butoxycarbonyl)ethoxy]methyl}methyl trifluoroacetamide (37)

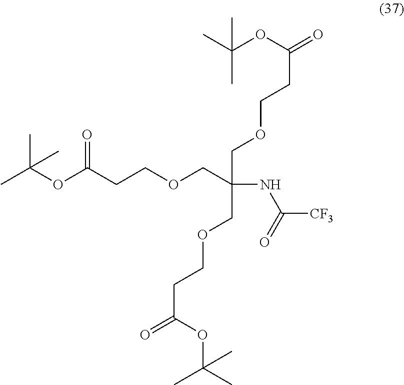

To a solution of tris{[2-(tert-butoxycarbonyl)ethoxy]methyl}methylamine (31) (10 mmol, 5.05 g) in methanol (30 mL) triethylamine (1 eq, 10 mmol, 1.39 mL) is added at rt. Ethyl trifluoroacetate (1.3 eq, 13 mmol, 1.55 mL) is slowly added over 20 min at rt. The reaction mixture is stirred overnight at rt. The solvent is evaporated, the residue is diluted with ethyl acetate (100 mL) and washed with a saturated solution of NaCl. The organic layer is dried over MgSO₄ and concentrated under reduced pressure. Flash chromatography (cyclohexane/ethyl acetate, 2/1→1/1) gives the desired compound (37).

ESI-MS: m/z 602.31 [M+H]⁺.

### Example 33

### N-Tris{[2-carboxyethoxy]methyl}methyl trifluoroacetamide (38)

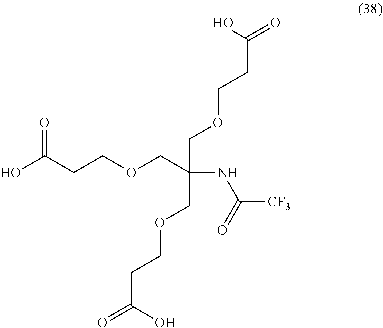

N-Tris{[2-(tert-butoxycarbonyl)ethoxy]methyl}methyl trifluoroacetamide (37) (4.81 g, 8 mmol) is stirred in 80 mL of 96% formic acid for 18 h. Formic acid is removed at reduced pressure at 50° C. to produce a colorless oil in quantitative yield.

ESI-MS: m/z 434.12 [M+H]⁺.

### Example 34

### N-Tris[2-(2-maleimidoethylaminocarbonyl)ethoxymethyl]methyl trifluoroacetamide (39)

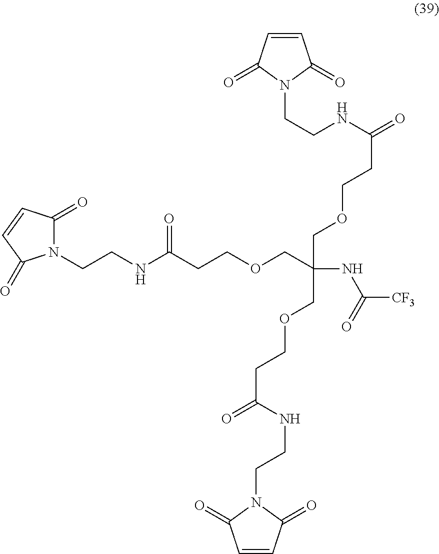

To a solution of compound (38) (433 mg, 1 mmol, 1 eq) and N-(2-aminoethyl)-maleimide trifluoracetate salt (1.8 g, 7 mmol, 7 eq) in DMF (15 mL) are successively added DIPEA (1.15 mL, 7 mmol, 7 eq), HOBT (1 M in NMP, 7 mL, 7 mmol, 7 eq) and DCC (1.44 g, 7 mmol, 7 eq) at rt. The resulting mixture is stirred overnight. The solvent is removed under reduced pressure and the mixture is diluted with 250 mL of ethyl acetate. The organic layer is washed with water, dried over MgSO₄ and evaporated under reduced pressure. Flash chromatography (CH₂Cl₂/methanol, 10/1→5/1) gives the desired compound (39). ESI-MS: m/z 800 [M+H]⁺.

### Example 35

### Tris[2-(2-maleimidoethylaminocarbonyl)ethoxymethyl]methylamine (40)

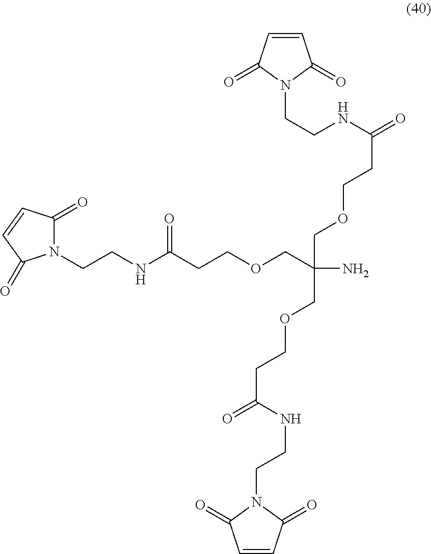

To a solution of compound (39) (480 mg, 0.6 mmol) in ethanol (15 mL) a solution of MeNH₂ (30% in EtOH, 30 mL) is added. The corresponding solution is stirred overnight at rt. A cloudy mixture is obtained. The solid is removed by filtration, and evaporation of the resulting clean solution affords the desired compound (40). No further purification is required. ESI-MS: m/z 704 [M+H]⁺.

### Example 36

### Tris{[2-(2-(2-CoA-S-succinimido)ethylaminocarbonyl)ethoxy]methyl}-methylamine (41)

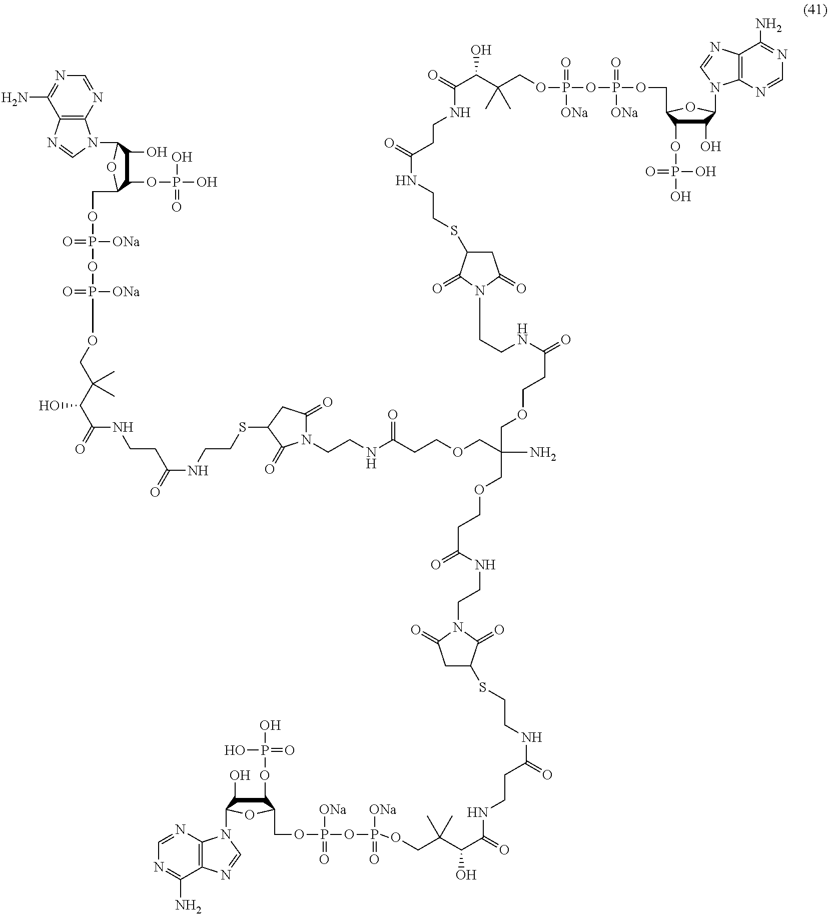

A solution of maleimide derivative (40) (9 mg, 1 eq, 13 μmol) in DMF (1 mL) is added to a solution of CoA-SH (35 mg, 3.3 eq, 43 μmol) in Tris-buffer (pH 7.5, 100 μL). The reaction mixture is shaken overnight at 31° C. The solvent is removed under vacuum and the crude mixture is purified via preparative HPLC. ESI-MS: m/z 3138 [M+H]⁺.

### Example 37

### N-Tris{[2-(2-(2-CoA-S-succinimido)ethylaminocarbonyl)ethoxy]methyl}methyl 3-[2-(2-maleimidoethyl)disulfanyl]propanoylamide (42)

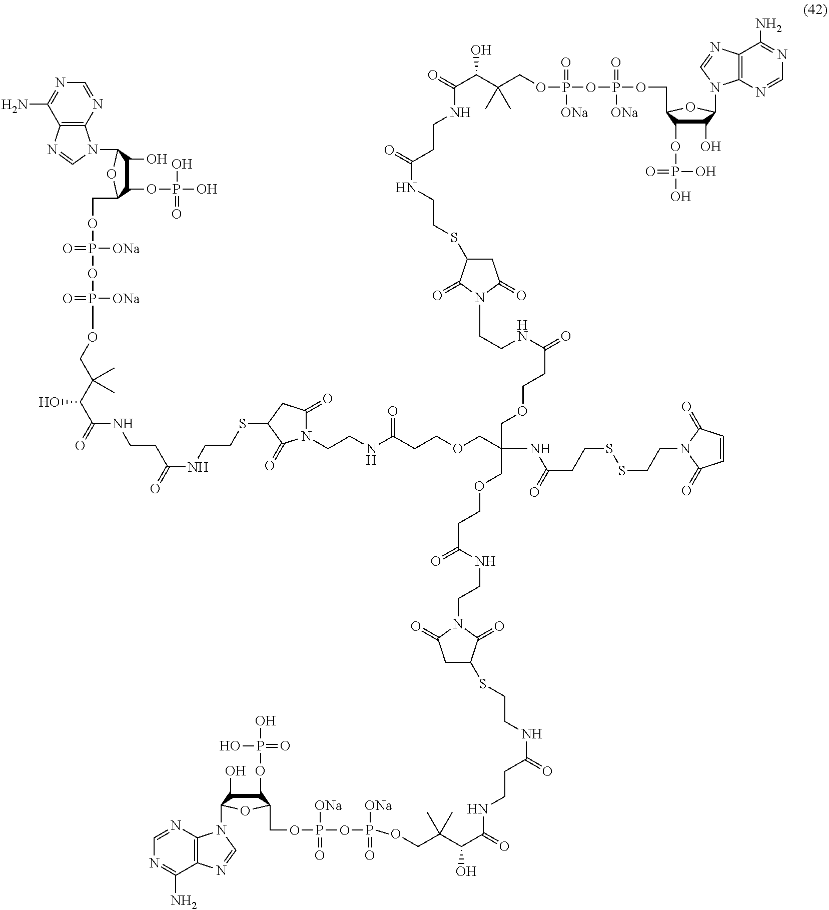

To a solution of 3-[2-(2-maleimidoethyl)disulfanyl]propanoic acid (36) (2 mg, 6.4 μmol) in DMF (1 mL) PYBOP (4 mg, 6.4 μmol) is added at rt. The solution is stirred at rt for 20 min. Compound (41) (20 mg, 6.4 μmol) and DIPEA (1 μL, 6.4 μmol) are added and the solution is heated at 50° C. for 5 min. The solution is stirred at rt overnight. The solvent is removed under vacuum and the crude mixture is purified via preparative HPLC.

ESI-MS: m/z 3382 [M+H]⁺.

### Example 38

### 4-Acetylbenzoic acid (E)-4-(maleimidomethyl)cyclohexanecarbonyl-hydrazone (43)

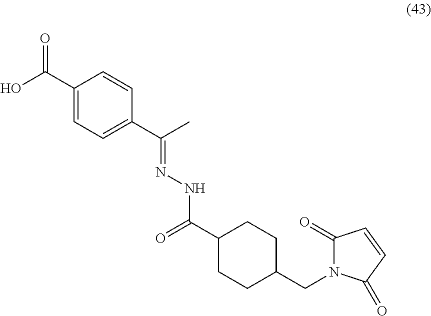

To a solution of 4-acetyl-benzoic acid (164 mg, 1 mmol) in methanol (10 mL) 4-((2,5-dioxo-2,5-dihydro-1H-pyrrol-1-yl)methyl)cyclohexanecarbohydrazide (364 mg, 1 mmol) and acetic acid (1 mL) are added at rt. The solution is heated under reflux for 6 h. The solvent is removed under reduced pressure. No further purification is required.

MS (ESI) m/z 398 [M+H]⁺.

### Example 39

### N-Tris{[2-(2-(2-CoA-S-succinimido)ethylaminocarbonyl)ethoxy]methyl}-methyl 4-acetylbenzamide (E)-4-(maleimidomethyl)cyclohexanecarbonyl-hydrazone (44)

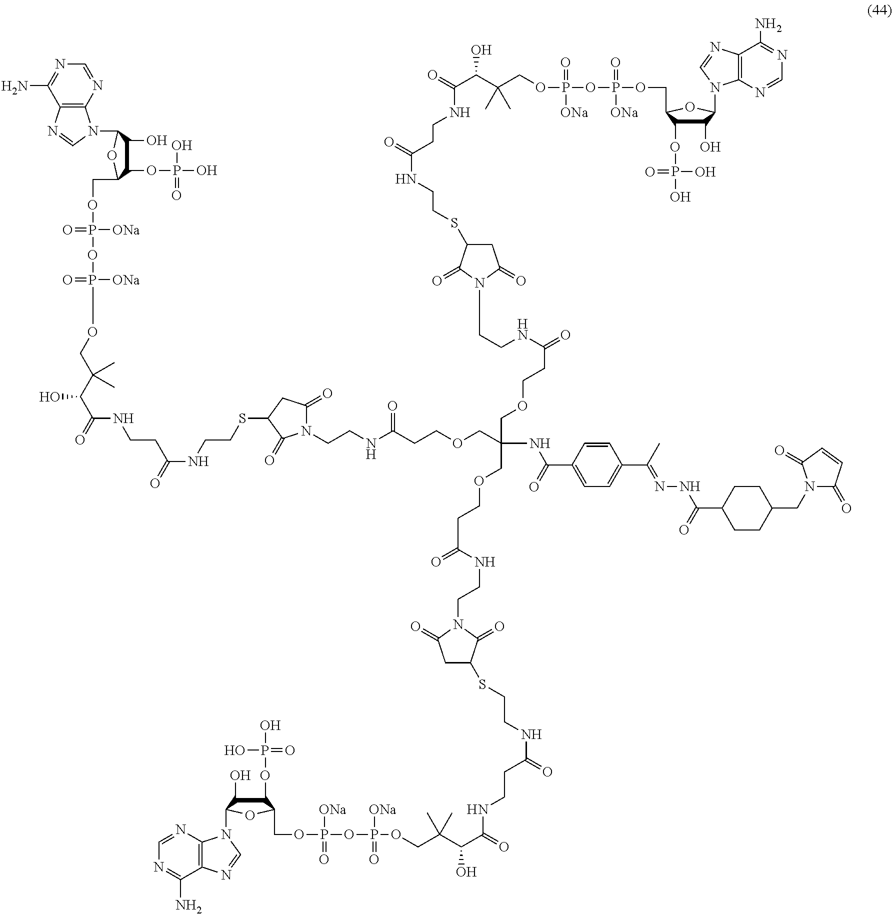

To a solution of compound (43) (4 mg, 0.01 mmol) in DMF (1 mL) PYBOP (5 mg, 0.01 mmol) is added at rt. The solution is stirred at rt for 20 min. Compound (41) (31 mg, 0.01 mmol) and DIPEA (1.6 μL, 0.01 mmol) are added and the solution is heated at 50° C. for 5 min. The solution is stirred at rt overnight. The solvent is removed under vacuum and the crude mixture is purified via preparative HPLC. ESI-MS: m/z 3518 [M+H]⁺.

1-10. (canceled)

11. The compound comprising coenzyme A (CoA) and one or more cargos having a formula: 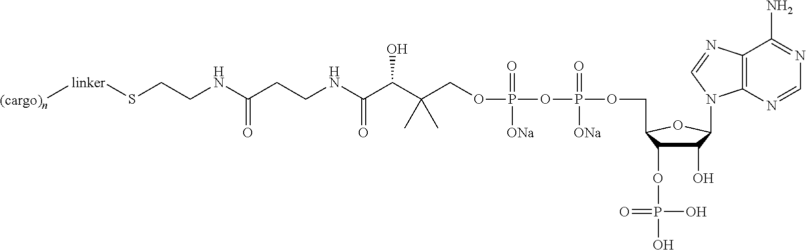
wherein n≧1 and the cargo is selected from a drug and a label detectable by a fluorescence detector, magnetic resonance imaging (MRI), positron emission tomography (PET) or scintigraphy, or a functional group which can be transformed into a drug or a detectable label, and wherein a plurality of cargos comprise the same or different drug, detectable label or functional group;
and wherein the linker may be branched or linear and further comprises at least one of
(i) 1 to 300 carbon atoms, wherein up to a third of the carbon atoms may be replaced by at least one of an oxygen, nitrogen or sulfur; and
(ii) double bonds, triple bonds, carbocycles or heterocycles, and may carry further substituents;

12. A compound according to claim 11, wherein the linker is a straight or branched chain alkylene group with 1 to 300 carbon atoms, wherein optionally
(a) one or more carbon atoms are replaced by oxygen;
(b) one or more carbon atoms are replaced by nitrogen or a nitrogen optionally substituted with a hydrogen atom to form —NH₃ or additionally a carbon substituted by an oxo to form an —NH—CO—; or, if two adjacent carbon atoms are replaced by nitrogen atoms, a hydrazine function —NH—NH— or a carbonylhydrazine function —NH—NH—CO—;
(c) one or more carbon atoms are replaced by oxygen, and adjacent carbons substituted by oxo to form —O—CO—;
(d) the bond between two adjacent carbon atoms is a double or a triple bond, to form —CH═CH— or —C≡C—, or the bond between a carbon and a nitrogen atom is a double bond representing an imine —C(R)═N— or a hydrazone —C(R)═N—NH—, or the bond between two adjacent nitrogen atoms is a double bond representing a diazo group —N═N—;
(e) one or more carbon atoms are replaced by a phenylene, a saturated or unsaturated cycloalkylene, a saturated or unsaturated bicycloalkylene, a bridging heteroaromatic or a bridging saturated or unsaturated heterocyclyl group;
(f) one or more carbon atoms are replaced by a sulfur atom, to form a thioether or, if two adjacent carbon atoms are replaced by sulfur atoms, a disulfide linkage —S—S—; or a combination of two or more optionally substituted alkylene and modified alkylene groups as defined in (a) to (f).

13. A compound comprising a protein directed to a target fused to an acyl carrier protein (ACP) or a fragment thereof, and linked to one or more of a drug entities, a label detectable by a fluorescence detector, magnetic resonance imaging (MRI), positron emission tomography (PET) or scintigraphy, and a functional group which can be transformed into a drug or a detectable label.

14. The compound according to claim 13, comprising:
wherein n≧1 and the cargo is selected from a drug and a label detectable by a fluorescence detector, magnetic resonance imaging (MRI), positron emission tomography (PET) or scintigraphy, or a functional group which can be transformed into a drug or a detectable label, and wherein a plurality of cargos comprise the same or different drug, detectable label or functional group;
and wherein the linker may be branched or linear and further comprises at least one of
(i) 1 to 300 carbon atoms, wherein up to a third of the carbon atoms may be replaced by at least one of an oxygen, nitrogen or sulfur; and
(ii) double bonds, triple bonds, carbocycles or heterocycles, and may carry further substituents;

15. The compound according to claim 14, wherein the protein directed to a target is a recombinant antibody fragment.

16. A pharmaceutical composition comprising a compound according to claim 15.

17. A method of treatment of cancer comprising: administering a compound according to claim 15 to a patient in need thereof.
